(19) United States
(12) Patent Application Publication
(10) Pub. No.: US 2018/0303776 A1
(43) Pub. Date: Oct. 25, 2018

(54) LIPOSOMAL REHYDRATION SALT FORMULATION FOR TREATMENT OF ALCOHOL RELATED DISORDERS

(71) Applicant: EINSOF BIOHEALTH LIMITED, Cork (IE)

(72) Inventors: Alcides NICASTRO, Santa Fe, AR (US); Alejandro Luis Barbarini, Santa Fe, AR (US); Gustavo M. Souss, Miami, FL (US)

(21) Appl. No.: 15/805,180

(22) Filed: Nov. 7, 2017

Related U.S. Application Data
(60) Provisional application No. 62/487,009, filed on Apr. 19, 2017.

(51) Int. Cl. A61K 31/164; A61K 9/127

(52) U.S. Cl. CPC A61K 31/164; A61K 9/127

(57) ABSTRACT

A liposomal rehydration salt formulation includes phospholipids at a concentration of about 1.0 g/L to 10.0 g/L, salts, water, oleoylethanolamide, and a percentage inclusion ratio of salts (salts retained within total salts/liposomes) of at least 50% and a sodium electrolyte of about 12 mEq/L to 90 mEq/L. The formulation has an actual osmolarity lower than 130 based on the at least 50% encapsulation of the salts and the liposomes comprise a particle diameter ranging from 200 nm to 500 nm.

### PRIORITY APPLICATION

This application is based on U.S. provisional patent application Ser. No. 62/487,009 filed Apr. 19, 2017, the disclosure which is hereby incorporated by reference in its entirety.

### RELATED APPLICATIONS

This application is related to the U.S. patent application Ser. No. 15/797,031 filed Oct. 30, 2017, which is a continuation-in-part application based on U.S. patent application Ser. No. 15/111,485 filed Jul. 14, 2016, which is based upon a U.S. national stage application as international Application No. PCT/ES2015/070003 filed Jan. 7, 2015, which claims priority from Argentina Patent Application No. P20140100123 filed Jan. 14, 2014, the disclosures which are hereby incorporated by reference in their entirety.

### FIELD OF THE INVENTION

The present invention relates to the technological field of improved oral rehydration salts. In particular, it relates to liposomal oral rehydration salts.

### STATE OF THE ART

References to oral rehydration salts in the form of liposomes are not abundant in literature. Several attempts to develop isolated products of this kind have been disclosed, which have not been successful.

It should be noted that U.S. Patent Publication No. 2005/0008685 (now abandoned) describes the use of liposomes for preparing oral rehydration salts. However, the percentage inclusion ratio of salts (salts retained within said liposomes/total salts) disclosed is 25%, which in spite of improving mouthfeel, still causes rejection by consumers or patients.

On the other hand, there are several reports on the benefits from administering liposomal rehydration salts, such as in “Absorption of Water and Electrolytes from a Liposomal Oral Rehydration Solution: An in vivo Perfusion Study of Rat Small Intestine” by P. K. Bardhan, A. S. M. Hamidur Rahman, Rifaat, and D. A. Sack—ICDDR,B: Centre for Health and Population Research, GPO Box 128, Dhaka 1000, Bangladesh, published in December 2003. This document makes reference to the improved mouthfeel and improved absorption mechanism of rehydration salts due to the presence of liposomes.

Salt concentration as recommended by the WHO for rehydration salts is the following:

| ORS | | | Concentration mmol/L | | | | |
|---|---|---|---|---|---|---|---|
| Function | Component | g/L | Glucose | Na+ | K+ | Cl− | Cit3− |
| Rehydration | Sodium | 2.6 | | 44.5 | | 44.5 | |
| salts | chloride | | | | | | |
| | Potassium | 1.5 | | | 20.1 | | |
| | Chloride | | | | | | |
| | Sodium | 2.9 | | 29.6 | | | 9.9 |
| | citrate | | | | | | |
| Sweetener | Glucose | 13.5 | 74.9 | | | | |

### DESCRIPTION OF THE DRAWINGS

FIG. 1 shows TEM (Transmission Electron Microscopy) images of a liposome sample of the present invention after the final stage of the preparation process.

FIG. 2 illustrates the diameter distribution of the liposomes of the present invention formulation, wherein the particle size distribution in a DLS (Dynamic Light Scattering) analysis is shown.

FIG. 3 illustrates a calibration curve for turbidity measurement.

FIG. 4 represents the evolution of body mass in male animals according to Example 9.

FIG. 5 represents the evolution of body mass in female animals according to Example 9.

FIG. 6 represents the evolution of hematocrit concentration in male animals according to Example 9.

FIG. 7 represents the evolution of hematocrit concentration in female animals according to Example 9.

FIG. 8 represents the evolution of sodium concentration (Natremia) (mmol/L) in male animals according to Example 9.

FIG. 9 represents the evolution of sodium concentration (Natremia) (mmol/L) in female animals according to Example 9.

FIG. 10 represents the evolution of potassium concentration (Kalemia) (mmol/L) in male animals according to Example 9.

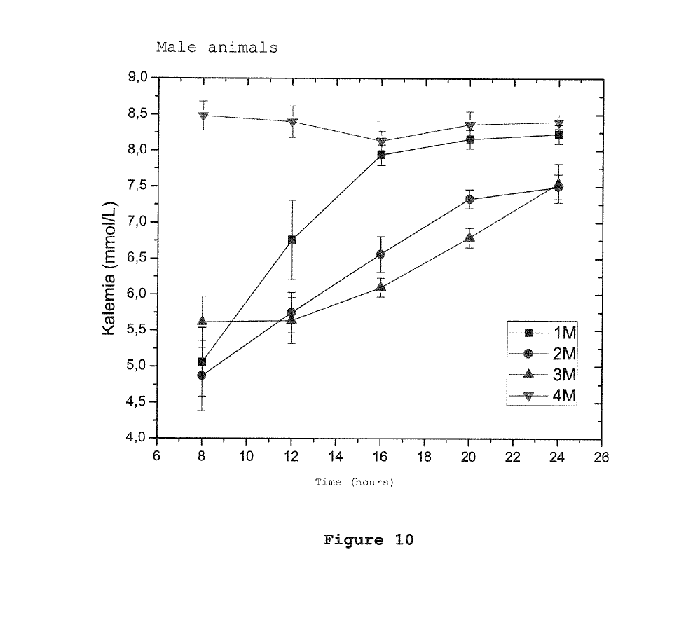

FIG. 11 represents the evolution of potassium concentration (Kalemia) (mmol/L) in female animals according to Example 9.

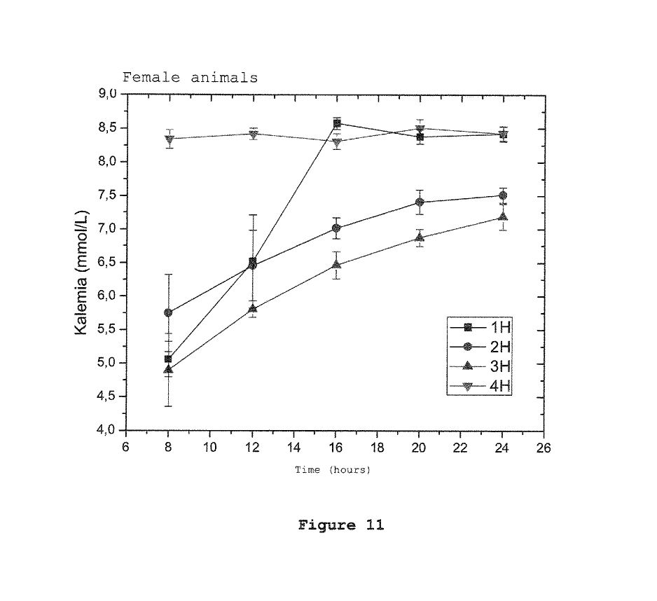

### SUMMARY OF THE INVENTION

A liposomal rehydration salt formulation comprises phospholipids at a concentration of about 1.0 g/L to 60.0 g/L, salts, water, and a percentage inclusion ratio of salts (salts retained within total salts/liposomes) of at least 40%, and a compound of general formula (I)

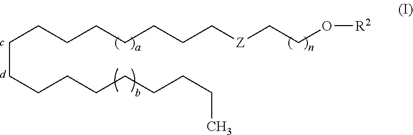

or any of its pharmaceutically acceptable salts, esters, tautomers, solvates and hydrates, or any combination thereof and wherein n is an integer ranging from 0 to 5 and a and b are determined by the following formula: 0≤(a+b)≤4, and Z is a group selected from —C(O)N(R0)-; —(R0)NC(O)—; —(O)CO—; OR; NR0; and S, and wherein R0 and R2 are independently selected from the group consisting of substituted or unsubstituted alkyl, hydrogen, substituted or unsubstituted (C1-C6) alkyl, substituted or unsubstituted (C1-C6) acyl, and aryl, and wherein up to eight hydrogen atoms of the compound may be substituted by methyl or a double bond, and the molecular bridge between c and d may be saturated or unsaturated. The formulation comprises an osmolality lower than 190 mmol/L.

The compound may comprise oleoylethanolamide and the liposomes may comprise a particle diameter ranging from 200 nm to 500 nm. The compound may be in the range of about 0.1 to 200 mg per daily dosage serving of the formulation and the compound has a percentage inclusion ratio of total compound/liposomes of at least about 40 percent.

In yet another example, a liposomal rehydration salt formulation comprises phospholipids at a concentration of about 1.0 g/L to 10.0 g/L, salts, water, oleoylethanolamide, and a percentage inclusion ratio of salts (salts retained within total salts/liposomes) of at least 50% and a sodium electrolyte of about 12 mEq/L to 90 mEq/L, wherein said formulation has an actual osmolarity lower than 130 based on the at least 50% encapsulation of the salts and the liposomes comprise a particle diameter ranging from 200 nm to 500 nm.

The oleoylethanolamide may be in the range of about 0.1 to 200 mg per daily dosage serving of the formulation and in another example, the oleoylethanolamide may be in the range of about 5 to 30 mg per daily dosage serving of the formulation. The oleoylethanolamide may have a percentage inclusion ratio of total compound/liposomes of at least about 40 percent. The sodium electrolyte may be from about 35 mEq/L to 55 mEq/L, and the formulation may comprise about 15 mEq/L to 25 mEq/L of potassium electrolyte.

The phospholipids may be selected from the group consisting of phosphatidylcholines (PCs), phosphatidylserines (PSs), phosphatidylethanolamines (PEs), phosphatidylglycerols (PGs), phosphatidylinositols (PIs), phosphatidic acids (PAs), and mixtures thereof and the formulation may further comprise an antioxidant selected from the group consisting of phytosterol, tocopherol, and mixtures thereof. The salts may be selected from the group consisting of sodium chloride at a concentration of 0.7 g/L to 2.8 g/L, potassium citrate at a concentration of 0.8 g/L to 2.5 g/L, sodium citrate at a concentration of 0.5 g/L to 2.9 g/L, and mixtures thereof. The formulation may further comprise about 10 g/L to 17 g/L of glucose and about 8.0 g/L to 15 g/L of at least one additional sugar.

A method of treating alcohol related disorders comprises orally administering a liposomal rehydration salt formulation, comprising phospholipids at a concentration of about 1.0 g/L to 60.0 g/L, oleoylethanolamide, salts, water, and a percentage inclusion ratio of salts (salts retained within total salts/liposomes) of at least 40%, and wherein said formulation comprises an osmolality lower than 190 mmol/L. The liposomes may comprise a particle diameter ranging from 200 nm to 500 nm, and about 0.1 to 200 mg of the oleoylethanolamide may be administered per daily dosage serving of the formulation. The oleoylethanolamide may have a percentage inclusion ratio of total compound/liposomes of at least about 40 percent.

### DETAILED DESCRIPTION

The liposomal rehydration salt formulation of the present invention contains phospholipid liposomes, preferably selected from the group consisting of phosphatidylcholines (PCs), phosphatidylserines (PSs), phosphatidylethanolamines (PEs), phosphatidylglycerols (PGs), phosphatidylinositols (PIs), phosphatidic acids (PAs), and mixtures thereof, at a concentration of less than 6% (W/V); and optionally an antioxidant selected from phytosterol, tocopherol, and mixtures thereof, at a concentration of 0.2 to 0.5% (W/V); water; salts selected from the group consisting of sodium chloride at a concentration of 0.7 to 2.8 g/l, potassium citrate at a concentration of 0.8 to 2.5 g/l, sodium citrate at a concentration of 0.5 to 2.9 g/l, and mixtures thereof; optionally, it may further comprise carbohydrates, among which glucose is preferred.

Intestinal salt absorption mechanisms are enterocyte co-transport systems. These systems involve carrying salts into the body along with other molecules, glucose being the most important among them. This is why rehydration salt formulations targeting hyponatremia, associated both with sports and acute diarrhea, are composed of a mixture of salts and glucose. Salt concentration should be higher than that of the body, so that glucose-mediated transport can be enabled by an osmotic gradient allowing for incorporation of salts through membranes. However, glucose intake is restricted by the calorie intake of this molecule.

Liposomes are nanoparticles consisting of a phospholipid bilayer, the same as cell membranes of enterocytes. Based on different mechanisms, liposomes (and all the contents carried in them) are highly capable of being absorbed by the small intestine cells, increasing bioavailability of the transported actives. Liposomal rehydration salt formulations aim at providing transport mechanisms of liposomes to the basic mechanisms of salts. In vivo tests have shown that an encapsulated ORS formulation having salt concentrations in accordance with WHO standards causes a 1.39-fold hydration increase in animals under normal conditions, as compared to the WHO recommended formula, and a 1.45-fold hydration increase in animals infected with cholera as compared to the WHO recommended formula (“Absorption of Water From a Liposomal Oral Rehydration Solution: an In Vivo Perfusion Study of Rat Small Intestine Exposed to Cholera Toxin” Gastroenterology—Volume 142, Issue 5, Supplement 1, Pages S-21, May 2012—Pradip K. Bardhan, Nasirul Islam, Rifat Faruqui).

In view of the above, one of the great advantages of the present invention relies on the use of lower carbohydrate concentrations, ranging from 0 to 6 g/l, which improves mouthfeel and tolerance to the formulation. Furthermore, it would be possible to replace glucose with a mixture of carbohydrates such as fructose, dextrose, high fructose corn syrup and mixtures thereof, and even with artificial sweeteners such as sucralose. Low glucose concentration is very important in sport drinks. It is even possible to accomplish efficient rehydration in the absence of glucose, which would allow the formulation to be consumed by diabetics.

In addition, and also due to lower glucose concentration, the formulation of the present invention exhibits reduced osmolality with respect to commercially-available formulations, also lower than 190 mmol/L, which accounts for the possibility of accomplishing efficient rehydration without running the risk of inducing hypernatremia in the patient.

Furthermore, one of the novel aspects of this invention is the fact that it significantly improves percentage inclusion ratio of salts (salts retained within said liposomes/total salts) with respect to the prior art. This ratio is at least 40%, preferably at least 50%, more preferably at least 52%. In a preferred alternative of the present invention, said percentage inclusion ratio is at least 56%. These inclusion ratio values have not been previously disclosed in the prior art, and they allow for the preparation of formulations containing lower salt concentrations with improved rehydration effects, as disclosed in the present invention. This inclusion ratio is achieved by using tangential ultrafiltration method. Although well-known, this method has never been employed to increase the ratio of oral rehydration salts encapsulated within liposomes to the total amount of the salts of the formulation, thereby solving the technical problem of rejection caused by oral rehydration salts due to their unpleasant taste.

It has been demonstrated in Example 4 that encapsulation of more than 50% of the salts in the formulation of the present invention causes unpleasant taste inherent to salts to be almost imperceptible. This facilitates consumption by children younger than 12 years, who represent the most affected population in terms of acute dehydration.

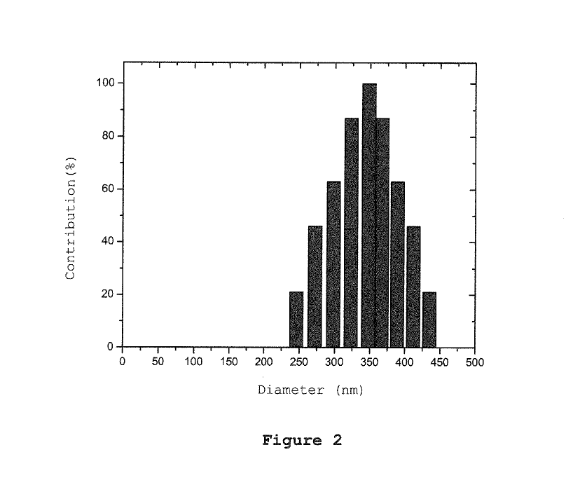

Furthermore, the liposomes of the formulation of the invention are produced such that the particle diameter ranges from 200 to 500 nm; preferably from 225 to 450 nm, as shown in FIG. 2.

The liposomal rehydration salt formulation of the present invention is an oral administration infusion for oral replacement of fluids and electrolyte salts in the treatment of dehydration caused by diarrhea and vomiting, prevention of severe dehydration, and maintenance of body electrolytes and liquids. The present invention may also be an oral administration infusion for use in sport activities.

The process for preparing the formulation of the invention comprises the following steps:

a. preparing an aqueous phase (AP) or buffer comprising sodium chloride, potassium citrate, sodium citrate dissolved in distilled water;

b. separately preparing an ethanol phase (EP), by dissolving said phospholipid at a concentration of 0.1 to 6% (W/V), and optionally an antioxidant at a concentration of 0.2 to 0.5% (W/V) in alcohol, preferably ethyl alcohol;

c. inducing formation of liposomes by injecting said EP into said AP at room temperature, while stirring;

d. subjecting the liposomal solution obtained in step “c” to a tangential ultrafiltration (TUF) concentration process, removing the buffer and maintaining the liposomes and their contents, thus reducing the volume at least by 10-fold;

e. subjecting the liposomal solution obtained in step “d” to a tangential ultrafiltration (TUF) concentration process, wherein ethanol is eliminated and the buffer is replaced with saline solution, and maintaining the liposomes and their contents.

In step “a”, said aqueous phase (AP) or buffer comprises sodium chloride at a concentration of 6 to 20 mmol/l, potassium citrate at a concentration of 1 to 12 mmol/l, sodium citrate at a concentration of 2 to 5 mmol/l, and distilled water.

In step “e” of the process of the present invention, said saline solution comprises a sodium concentration of 12 to 50 mmol/l, a potassium concentration of 3 to 36 mmol/l, a chloride concentration of 15 to 40 mmol/l, a citrate concentration of 8 to 17 mmol/l, and it further comprises glucose at a concentration of 17 to 45 mmol/l.

Furthermore, the AP:EP volume ratio in step “c” is at least 10:1; preferably at least 10:0.5; more preferably at least 10:0.4.

The process of the present invention comprises a perpendicular flow process, wherein the ethanol phase is added on the aqueous phase by perpendicular coupling to the flow of the former, and with a linear velocity ratio REP/RAP of no more than 1/200.

### EXAMPLES

### Example 1

### Preparation of the Liposomal Rehydration Salt Formulation of the Invention

### a) Preparation of the Ethanol Phase (EP)

25 g of purified soybean phosphatidylcholine and 0.5 L of ethanol are added, heated to 65° C., and stirred until completely dissolved. A total amount of 2.5 g of mixed tocopherols (ascorbyl palmitate and D-Alpha-Tocopherol) is added as antioxidant. The solution is left to rest until it reaches room temperature.

### b) Preparation of the Saline Aqueous Phase (AP)

4.33 g Sodium chloride, 3.42 g Potassium citrate, and 4.83 g Sodium citrate are dissolved in 4.5 L water and stirred at room temperature until completely dissolved.

### c) Production of Liposomes

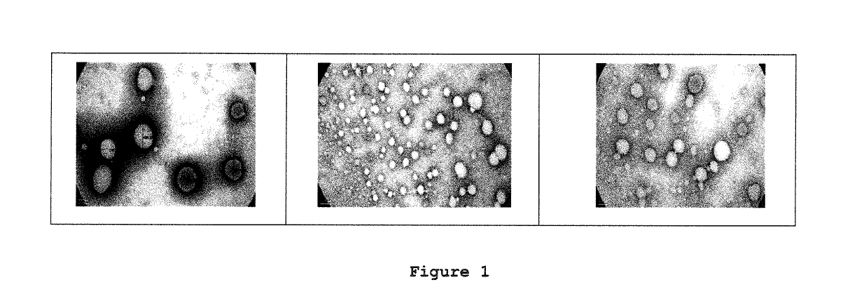

0.5 L of Ethanol phase is slowly added on 4.5 L of Aqueous phase under continuous circular stirring. This may be also performed by means of a Cross-Flow or Perpendicular Flow process, wherein the Ethanol phase is added on the Aqueous phase by perpendicular coupling to the flow of the former, and with a linear velocity ratio, REP/RAP, of no more than 1/200. FIG. 1 shows liposomes formed with both processes. FIG. 2 shows the results of particle size distribution in DLS (Dynamic Light Scattering) analysis.

### d) Increasing Encapsulation Efficiency

Ultrafiltration without recirculation, by tangential flow, is carried out so as to remove the aqueous phase solutes that are not trapped in the liposomes. This process is completed after removing 90% volume of the previous liposomal dispersion.

### e) Buffer Substitution

Ultrafiltration by tangential flow is carried out to remove ethanol from the liposomal salt solution. While the process is conducted, the solution is fed at a speed equal to the permeation speed with an aqueous solution of Sodium chloride (1.05 mg/ml), Potassium citrate (0.83 mg/ml), Sodium citrate (1.17 mg/ml) and Glucose (6.75 mg/ml).

The liposomal rehydration salt formulation of the present invention is thereby obtained, said formulation having the following features:
Percentage inclusion ratio of salts (salts retained within liposomes/total salts) of 56.48%
Chloride concentration: 39.7 mmol/L
Citrate concentration: 16 mmol/L
Potassium concentration: 17.9 mmol/L
Sodium concentration: 69.7 mmol/L
Glucose concentration: 33.0 mmol/L

### Example 2

### Process for Preparing the Present Invention Formulation with a Percentage Inclusion Ratio of Salts of 56%

Stage a

A solution of 4.5 L distilled water with salts is prepared at the following concentration:

| | Concentration (mmol/L) | | | | |
|---|---|---|---|---|---|
| | Glucose | Na | K | Cl | Cit |
| Sodium chloride | | 14.82 | | 14.82 | |
| Potassium citrate | | | 6.70 | | 2.23 |
| Sodium citrate | | 11.23 | | | 3.74 |
| Glucose | — | | | | |

Stage b

Separately, a solution of Phosphatidylcholine in 500 ml of 5% ethyl alcohol (W/V) is prepared.

Stage c

Formation of liposomes is induced by injecting the ethanol solution into the aqueous phase while stirring. Then 15% of the salts are encapsulated; therefore, internal and external salt concentrations are as follows:

| | Internal | External |
|---|---|---|
| Na | 3.91 | 22.14 |
| K | 1.00 | 5.70 |
| Cl | 2.23 | 12.60 |
| Cit | 0.895 | 5.074 |
| Glucose | 0 | 0 |

Stage d

Five (5) liters of liposomal ORSs are subjected to a tangential ultrafiltration concentration process. This process allows for removing the buffer without eliminating the liposomes and their contents. This process is performed until the volume is reduced by 10-fold. At the end of the process, 500 ml of liposomal salts having the following concentration is obtained.

| | Internal | External |
|---|---|---|
| Na | 39.1 | 22.14 |
| K | 10.0 | 5.70 |
| Cl | 22.3 | 12.60 |
| Cit | 8.95 | 5.074 |
| Glucose | 0 | 0 |

Stage e

At this stage, the buffer is substituted by using the TUF process again. In this case, the total volume is reduced by 10-fold, and replaced with an aqueous solution with the following salt concentration.

| | Concentration (mmol/L) |
|---|---|
| Na | 31.57 |
| K | 8.13 |
| Cl | 17.96 |
| Cit | 7.24 |
| Glucose | 40.70 |

Accordingly, 500 ml of a solution of liposomal ORSs having the following salt concentration is obtained.

| | Internal | External | TOTAL |
|---|---|---|---|
| Na | 39.1 | 30.64 | 69.74 |
| K | 10.0 | 7.88 | 17.88 |
| Cl | 22.3 | 17.45 | 39.75 |
| Cit | 8.95 | 7.02 | 15.97 |
| Glucose | 0 | 33.03 | 33.03 |

The so obtained formulation exhibits a salt concentration equal to that of the formulation recommended by the WHO, with an encapsulation efficiency of about 56.05%. Other features recommended by the WHO and UNICEF in their joint statement issued in May 2004 and accomplished in this invention are reduced glucose content and lower osmolality.

The liposomal rehydration salt formulation of the present invention is thereby obtained, said formulation having the following features:
Percentage inclusion ratio of salts (salts retained within
liposomes/total salts) of 56.05%
Chloride Concentration: 39.75 mmol/l
Citrate Concentration: 15.97 mmol/l
Potassium Concentration: 17.88 mmol/l
Sodium Concentration: 69.74 mmol/l
Glucose Concentration: 33.03 mmol/l

### Example 3

### Encapsulation Efficiency Using the Barium Sulphate Turbidity Method

Two phosphatidylcholine ethanol solutions are prepared, one of them named “FE1”, which has a concentration of 2% Phosphatidylcholine (the same as the one used in the TLEC formulation of U.S. Patent Publication No. 2005/0008685), and the other named “FE2”, with a concentration of 5% Phosphatidylcholine (the same as the one used in the present invention). Separately, an aqueous solution (AP) of 56.23 mM ammonium sulphate is prepared (this concentration reproduces the ionic strength of the WHO rehydration salts).

Two liposomal solutions are then prepared using the ethanol phase injection method, in which 10 ml FE1 and FE2 are separately injected in two fractions of 90 ml AP under magnetic stirring at 300 rpm and 25° C. Consequently, 100 ml of two liposomal formulations are obtained:
*1:10 (v/v) FE1:AP (LIPO-1)
*1:10 (v/v) FE2:AP (LIPO-2)

The LIPO-2 formulation was subjected to a tangential ultrafiltration process using a hollow fiber cartridge with a 300 KD cut off, without feedback. Ultrafiltration continued until reducing the volume by 10-fold. This is the process we carry out in our invention in order to obtain higher encapsulation efficiency.

Samples are taken from both final solutions.

Then, 10 ml of each formulation (LIPO-1 and LIPO-2) is taken and ultrafiltered by using the same system but feeding back each formulation with 126 mM sucrose aqueous buffer. Thus, the sulphate ions non-encapsulated into liposomes are eliminated from each solution and substituted with a solution having the same osmolality in order to ensure integrity of the liposomal membranes.

Then 5 ml of each formulation is taken before and after the ultrafiltration process, and 10% surfactant Triton X-100 is added to each of them, in order to break the lipid membranes. This solution is kept under stirring at 25° C. for 1 hour.

### Turbidity Measurement

Soluble sulphates precipitate in the presence of barium chloride in the form of barium sulphate (BaSO4) as a white solid. Measurement of tance reduction as a consequence of the presence of barium sulphate, to a certain wave length in a UV/Vis spectrometer allows for determination of the sulphate ion concentration in aqueous solution.

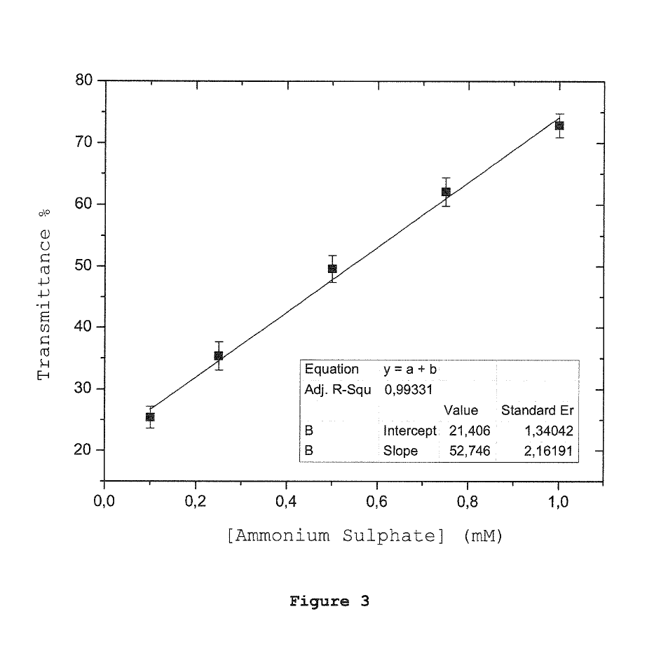

For experimental determination, a calibration curve was plotted by using 50 ml ammonium sulphate patterns with the following concentrations: 0.1 mM, 0.25 mM, 0.5 mM, 0.75 mM and 1 mM. An excess of 31.23 mg (3 mM) barium chloride was added to each solution. Then, transmittance of each solution was determined in triplicate by using an UV/VIS spectrometer (Jenway 7315). The calibration curve is shown in FIG. 3.

Then, 0.1 ml of the unknown LIPO-1 solution before ultrafiltration and 1 ml of the solution after ultrafiltration were taken, both already treated with surfactant, and the same excess of barium chloride was added (3 mM). Transmittance of the sample before ultrafiltration was 49.22%±0.17%, corresponding to [Sulphate]=0.5273 mM. Taking dilutions into account, the total sulphate concentration before ultrafiltration is 52.73 mM. Transmittance of the sample after ultrafiltration was 48.25%±0.32%, corresponding to [Sulphate]=0.5091 mM. Taking dilutions into account, the salt concentration after ultrafiltration is 5.091 mM. This indicates that the ratio of sulphate encapsulated in the formulation produced with the concentration of Phosphatidylcholine of U.S. Patent Publication No. 2005/0008685 was 10.59%.

Likewise, 0.1 ml of the unknown LIPO-2 solution before ultrafiltration, and 0.1 ml of the solution after ultrafiltration were taken, both already treated with surfactant, and the same excess of barium chloride was added (3 mM). Transmittance of the sample before ultrafiltration was 73.19%±0.19%, corresponding to [Sulphate]=0.9818 mM. Taking dilutions into account, the total sulphate concentration before ultrafiltration is 98.18 mM. Transmittance of the sample after ultrafiltration was 50.66%±0.24%, corresponding to [Sulphate]=0.5545 mM. Taking dilutions into account, the salt concentration after ultrafiltration is 55.45 mM. This indicates that the ratio of sulphate encapsulated in the formulation of the present invention was 56.48%.

Note: The results are directly proportional to the encapsulated rehydration salts, since all the salts have similar and increased water solubility. Therefore, the encapsulation level by the method of liposome formation by ethanol phase injection is statistical, and it will be similar in compounds with similar water solubility.

### Example 4

### Multicenter, Randomized and Single-Blind Mouthfeel Assay

### Liposomal Rehydration Salts

### Samples:

Formula A: Liposomal rehydration salt formulation of example 3 of the present invention.

Formula B: Liposomal rehydration salts according to example 3 of U.S. Patent Publication No. 2005/0008685 A1.

### Methodology

Healthy individuals from 21 to 40 years of age were recruited. Those individuals with cardiac or renal diseases, diabetics, individuals who had suffered from diarrhea the month prior to the assay, individuals affected by rhinitis, or individuals under antibiotic or iron treatment were excluded from the assay.

The screening of the individuals took place in four different shopping malls in the city of Santa Fe, Argentina. After explaining the test to the individuals and having them signed their consent (either by themselves or by their parents or legal guardians in case of underage people), the individuals were randomized. Randomization indicates the order in which the two formulations would be tasted. In order to get familiar with this type of flavors, the individuals took a little sip of the two formulations and then rinsed their mouths with water and a piece of salt-free bread. Thereafter, they tasted the two formulations in the order indicated by randomization, and they were asked to indicate the formulation of their preference. The same test was repeated twice with both formulations, after a new mouth rinse with water and pieces of bread. They were offered each formulation in amounts of less than 20 ml in total, inside red plastic glasses (to avoid color influence on the decision). The formulations were administered at room temperature, without any refrigeration.

Each individual tasted both formulations repeatedly (twice the first tasting and twice the second tasting); to corroborate consistency both times each tasting took place, kappa(k) statistic was used (URL: www.graphpad.com/quickcalcs/kappa2.cfm) as well as a 95% CI.

### Results

120 individual were studied, out of which 4 individuals did not meet the inclusion criteria (out of age), so the final test cohort consisted of 116 individuals with an average of 30-32 years old. The distribution of the individuals per shopping mall was similar: between 27 and 30 per each shopping mall. 59 individuals were female (50.9%).

Regarding the results obtained, we found very high consistency between the scores of the 2 tests with the same formulations, both in the first tasting (k=0.91; 95% CI: 0.85-0.98), and the second tasting (k=0.87; 95% CI: 0.80-0.94). Therefore, in the statistical analysis, it was decided to use the results corresponding to the second time each of the two tastings was scored.

Out of the 116 individuals, 97 individuals preferred the taste of formula A, 2 preferred the taste of formula B. 17 individuals were not certain as to which they preferred, so they were not counted.

### Example 5

### Process for Preparing the Formulation of the Present Invention with a Percentage Inclusion Ratio of Salts of 56% (for Sport Activities)

Stage a

A solution of 4.5 L distilled water is prepared with salts at the following concentration:

| | Concentration (mmol/L) | | | | |
|---|---|---|---|---|---|
| | Glucose | Na | K | Cl | Cit |
| Sodium chloride | | 6.01 | | 6.01 | |
| Potassium citrate | | | 3.86 | | 1.29 |
| Sodium citrate | | 6.02 | | | 2.01 |
| Glucose | — | | | | |

Stage b

On the other hand, a solution of phosphatidylethanolamine in 500 ml of 4% Ethyl Alcohol (W/V) is prepared.

Stage c

Liposome formation is induced by injecting the ethanol solution into the aqueous phase while stirring. Here, 15% of the salts are encapsulated. Therefore, the internal and external salt concentrations are the following:

| | Internal | External |
|---|---|---|
| Na | 1.57 | 10.46 |
| K | 0.50 | 3.36 |
| Cl | 0.78 | 5.23 |
| Cit | 0.49 | 2.81 |
| Glucose | 0 | 0 |

Stage d

The Five (5) liters of liposomal ORSs are subjected to a tangential ultrafiltration (TUF) concentration process. This process allows for removing the buffer without eliminating the liposomes and their contents. This process is carried out until reducing the volume by 10-fold. At the end of the process, 500 ml liposomal salts having the following concentration are obtained.

| | Internal | External |
|---|---|---|
| Na | 15.7 | 10.56 |
| K | 5.04 | 3.46 |
| Cl | 7.84 | 5.23 |
| Cit | 4.90 | 2.81 |
| Glucose | 0 | 0 |

Stage e

At this stage, buffer substitution is performed, again with the TUF process. In this case, the total volume is reduced by 10-fold and replaced with an aqueous solution having the following salt concentration:

| | Concentration (mmol/L) |
|---|---|
| Na | 12.56 |
| K | 3.65 |
| Cl | 5.65 |
| Cit | 3.04 |
| Glucose | 17.80 |

This buffer further contains Stevia (Reb A 97—PureCircle) at a concentration of 0.15 g/L; Sucrose at a concentration of 28.5 g/L; Citric Acid at a concentration of 3.6 g/L; and Natural Flavors at a concentration of 1.5 g/L.

Accordingly, 500 ml of a liposomal ORS solution is obtained, containing 40 g/l phospholipid, with the following salt concentration:

| | Internal | External | TOTAL |
|---|---|---|---|
| Na | 15.7 | 12.35 | 28.05 |
| K | 5.04 | 3.62 | 8.66 |
| Cl | 7.84 | 5.61 | 13.45 |
| Cit | 4.90 | 3.02 | 7.92 |
| Glucose | 0 | 16.02 | 16.02 |

The formulation of the present example is useful for people in need of hydration due to sun exposure, illness, pregnancy, travel fatigue, hangover, mental stress, strenuous work, or just living an active life. It may be produced with orange, strawberry, apple, pear, blueberry, raspberry flavors, among others.

### Example 6

### Process for Preparing the Formulation of the Present Invention with a Percentage Inclusion Ratio of Salts of 56%

### Pediatric Rehydration Formulation.

Stage a

A solution of 4.5 L distilled water is prepared with salts at the following concentration:

| | Concentration (mmol/L) | | | | |
|---|---|---|---|---|---|
| | Glucose | Na | K | Cl | Cit |
| Sodium chloride | | 14.82 | | 14.82 | |
| Potassium citrate | | | 6.70 | | 2.23 |
| Sodium citrate | | 11.23 | | | 3.74 |
| Glucose | — | | | | |

Stage b

On the other hand, a solution of phosphatidylserine in 500 ml of 3% Ethyl alcohol (W/V) is prepared.

Stage c

Liposome formation is induced by injecting the ethanol solution into the aqueous phase while stirring. Here, 15% of the salts are encapsulated. Therefore, the internal and external salt concentrations are the following:

| | Internal | External |
|---|---|---|
| Na | 3.91 | 22.14 |
| K | 1.00 | 5.70 |
| Cl | 2.23 | 12.60 |
| Cit | 0.895 | 5.074 |
| Glucose | 0 | 0 |

Stage d

The Five (5) liters of Liposomal ORSs are subjected to a tangential ultrafiltration concentration process. This process allows for removing the buffer without eliminating the liposomes and their contents. This process is carried out until the volume is reduced by 10-fold. At the end of the process 500 ml of liposomal salts having the following concentration is obtained.

| | Internal | External |
|---|---|---|
| Na | 39.1 | 22.14 |
| K | 10.0 | 5.70 |
| Cl | 22.3 | 12.60 |
| Cit | 8.95 | 5.074 |
| Glucose | 0 | 0 |

Stage e

At this stage, buffer substitution is carried out, again with the TUF process. In this case, the total volume is reduced by 10-fold and replaced with an aqueous solution having the following salt concentration:

| | Concentration (mmol/L) |
|---|---|
| Na | 31.57 |
| K | 8.13 |
| Cl | 17.96 |
| Cit | 7.24 |
| Glucose | 40.70 |

This buffer further contains Sucralose at a concentration of 0.12 g/L; high fructose corn syrup (55° Brix) at a concentration of 33.3 g/L; Citric Acid at a concentration of 4.0 g/L; and Natural Flavors at a concentration of 1.7 g/L.

Accordingly, 500 ml of a liposomal ORS solution with 30 g/l phosphatidylserine and the following salt concentration is obtained:

| | Internal | External | TOTAL |
|---|---|---|---|
| Na | 39.1 | 30.64 | 69.74 |
| K | 10.0 | 7.88 | 17.88 |
| Cl | 22.3 | 17.45 | 39.75 |
| Cit | 8.95 | 7.02 | 15.97 |
| Glucose | 0 | 33.03 | 33.03 |

The formulation of the present example is useful for children suffering from vomiting or diarrhea under the risk of dehydration, and it may be produced with orange, strawberry, apple, pear, blueberry, raspberry flavors, among others.

### Example 7

### Process for Preparing the Formulation of the Present Invention with a Percentage Inclusion Ratio of Salts of 56%

Stage a

A solution of 4.5 L distilled water is prepared with salts at the following concentration:

| | Concentration (mmol/L) | | | | |
|---|---|---|---|---|---|
| | Glucose | Na | K | Cl | Cit |
| Sodium chloride | | 6.01 | | 6.01 | |
| Potassium citrate | | | 3.86 | | 1.29 |
| Sodium citrate | | 6.02 | | | 2.01 |
| Glucose | — | | | | |

Stage b

On the other hand, a solution of Phosphatidylcholine in 500 ml of 5% Ethyl alcohol (W/V) is prepared.

Stage c

Liposome formation is induced by injecting the ethanol solution into the aqueous phase while stirring. Here, 15% of the salts are encapsulated. Therefore, the internal and external salt concentrations are the following:

| | Internal | External |
|---|---|---|
| Na | 1.57 | 10.46 |
| K | 0.50 | 3.36 |
| Cl | 0.78 | 5.23 |
| Cit | 0.49 | 2.81 |
| Glucose | 0 | 0 |

Stage d

The five (5) liters of Liposomal ORSs are subjected to a tangential ultrafiltration concentration process. This process allows for removing the buffer without eliminating the liposomes and their contents. This process is carried out until the volume is reduced by 10-fold. At the end of the process, 500 ml of liposomal salts having the following concentration is obtained.

| | Internal | External |
|---|---|---|
| Na | 15.7 | 10.46 |
| K | 5.04 | 3.46 |
| Cl | 7.84 | 5.23 |
| Cit | 4.90 | 2.81 |
| Glucose | 0 | 0 |

Stage e

At this stage, buffer substitution is carried out, again with the TUF process. In this case, the total volume is reduced by 10-fold and replaced with an aqueous solution having the following salt concentration:

| | Concentration (mmol/L) |
|---|---|
| Na | 12.56 |
| K | 3.65 |
| Cl | 5.65 |
| Cit | 3.04 |
| Glucose | 17.80 |

This buffer further contains Stevia (Reb A 97—PureCircle) at a concentration of 0.13 g/L; Sucrose at a concentration of 22.2 g/L; Citric Acid at a concentration of 3.4 g/L; and Natural Flavors at a concentration of 1.5 g/L.

Accordingly, 500 ml of a liposomal ORS solution is obtained having the following salt concentration:

| | | | TOTAL |
|---|---|---|---|
| | Internal | External | (mmol/L) |
| Na | 15.7 | 12.35 | 28.05 |
| K | 5.04 | 3.62 | 8.66 |
| Cl | 7.84 | 5.61 | 13.45 |
| Cit | 4.90 | 3.02 | 7.92 |
| Glucose | 0 | 16.02 | 16.02 |

This formulation may be suitable for consumption by sportspeople.

### Example 8

### Process for Preparing the Formulation of the Present Invention with a Percentage Inclusion Ratio of Salts of 56% for High-Performance Sportspeople

Stage a

A solution of 4.5 L distilled water is prepared with salts at the following concentration:

| | Concentration (mmol/L) | | | | |
|---|---|---|---|---|---|
| | Glucose | Na | K | Cl | Cit |
| Sodium chloride | | 6.01 | | 6.01 | |
| Potassium citrate | | | 3.86 | | 1.29 |
| Sodium citrate | | 6.02 | | | 2.01 |
| Glucose | — | | | | |

Stage b

On the other hand, a solution of phosphatidylinositol in 500 ml of 5% Ethyl alcohol (W/V) is prepared.

Stage c

Liposome formation is induced by injecting the ethanol solution into the aqueous phase while stirring. Here, 15% of the salts are encapsulated. Therefore, the internal and external salt concentrations are the following:

| | Internal | External |
|---|---|---|
| Na | 1.57 | 10.46 |
| K | 0.50 | 3.36 |
| Cl | 0.78 | 5.23 |
| Cit | 0.49 | 2.81 |
| Glucose | 0 | 0 |

Stage d

The 5 Liters of Liposomal ORSs are subjected to a (TUF) tangential ultrafiltration concentration process. This process allows for removing the buffer without eliminating the liposomes and their contents. This process is carried out until the volume is reduced by 10-fold. At the end of the process, 500 ml of liposomal salts having the following concentration is obtained.

| | Internal | External |
|---|---|---|
| Na | 15.7 | 10.46 |
| K | 5.04 | 3.46 |
| Cl | 7.84 | 5.23 |
| Cit | 4.90 | 2.81 |
| Glucose | 0 | 0 |

Stage e

At this stage, buffer substitution is carried out, again with the TUF process. In this case, the total volume is reduced by 10-fold and replaced with an aqueous solution having the following salt concentration:

| | Concentration (mmol/L) |
|---|---|
| Na | 12.56 |
| K | 3.65 |
| Cl | 5.65 |
| Cit | 3.04 |
| Glucose | 0 |

This buffer further contains high fructose corn syrup (55° Brix) at a concentration of 3.22 g/L; Vitamin B1 at a concentration of 0.002 g/L; Vitamin B5 at a concentration of 0.011 g/L; Vitamin B6 at a concentration of 0.011 g/L; Citric Acid at a concentration of 3.6 g/L; and Natural Flavors at a concentration of 1.5 g/L.

Accordingly, 500 ml of a liposomal ORS solution with 50 g/l phosphatidylinositol and the following salt concentration is obtained:

| | Internal | External | TOTAL |
|---|---|---|---|
| Na | 15.7 | 12.35 | 28.05 |
| K | 5.04 | 3.62 | 8.66 |
| Cl | 7.84 | 5.61 | 13.45 |
| Cit | 4.90 | 3.02 | 7.92 |
| Glucose | 0 | 16.02 | 16.02 |

This formulation may be suitable for consumption by high-performance sportspeople.

### Example 9

### Preclinical Assay of the Rehydration Salt Formulation of the Present Invention

A batch of pediatric liposomal rehydration salts of Example 6 of the present invention as a finished product is compared to commercial product Pedialyte (Abbott Laboratories) taken as reference substance. Said comparison encompassed the development of an osmotic diarrhea model in rats for efficiency evaluation.

### Experimental Design:

An osmotic diarrhea experimental model was developed as described in Wapnir et al., 1988, 1991 (Am. J. Clin. Nutr. 1988; 4784-90; J. Pediatr. 1991; 118:S53-61). Four experimental animal groups were used, each consisting of 10 animals (5 male and 5 female animals). Groups 1, 2 and 3 were induced diarrhea by replacing the water for an oral solution of 50% magnesium citrate (USP XXII) for 5 days. Group 4 was not induced diarrhea and was allowed to drink water during said period. Once induction was completed, Group 1 was treated with the test substance; Group 2 was treated with the reference substance; Group 3 received physiological solution; while Group 4 was not treated at all. Body weight, Natremia, Kalemia, and Hematocrit variables were analyzed both during treatment and 12 hours after completion. Young female and male Wistar rats with genetic certification were used. They were divided into subgroups, placed into jails, and identified with a correlative integer number.

The animals were kept under controlled ambient conditions: temperature between 22±3° C., controlled photoperiod (12 hs light/12 hs darkness) and free access to commercial food and water. MicroVENT rack systems provided by Allentown Inc., European Type IIIH (POE GC-065) models, were employed.

Forty (40) animals divided into four experimental groups (each group comprising 5 male and 5 female animals) were used.

Group 1: (5 male and 5 female animals). It was distributed into 2 subgroups: 1-M; 1-F, each consisting of 5 animals of the same sex. These animals were subjected to osmotic diarrhea induction and treated with the test substances.

Group 2: (5 male and 5 female animals). It was distributed into 2 subgroups: 2-M; 2-F, each consisting of 5 animals of the same sex. These animals were subjected to osmotic diarrhea induction and treated with the reference substance.

Group 3: (5 male and 5 female animals). It was distributed into 2 subgroups: 3-M; 3-F, each consisting of 5 animals of the same sex. These animals were subjected to osmotic diarrhea induction and treated with physiological solution.

Group 4: (5 male and 5 female animals). It was distributed into 2 subgroups: 4-M; 4-F, each consisting of 5 animals of the same sex. These animals did not receive any treatment.

### Treatment:

### Osmotic Diarrhea Induction:

In groups 1, 2, and 3, an osmotic diarrhea experimental model was developed as described in Wapnir et al., 1988, 1991 (Am. J. Clin. Nutr. 1988; 4784-90; J. Pediatr. 1991; 118:S53-61).

### Test Substances:

Liposomal Rehydration Salts—Pediatric Formulation of Example 6 of the present invention.

### Reference Substance:

Rehydration Salts Pedialyte—Pediatric Formulation manufactured by Abbott Laboratories.

### Dosage and Administration:

Groups 1, 2, and 3 were orally administered a total dose of 125 ml/kg/day of the different test substances, distributed in 12 doses at a one-hour-interval between each other. The dose was selected taking into account the dosing instructions of Pedialyte according to which doses of 100 to 150 ml/kg are recommended. The dosage volume of administration was calculated according to the average weight of the male and female rats obtained on Day 5 during the morning, at the time magnesium citrate solution was removed and treatment was initiated, thereby determining differential doses for male and female rats.

The volume corresponding to each dosage was calculated according to the following formula:

$$V ( \frac{ml}{animal} ) = \frac{P \times 125}{12 \times 1000}$$

Wherein P is the average weight in grams, either of the male or female rats, as applicable.

Dose administration took place every hour beginning at 9 A.M. on Day 5.

### Hematocrit Determination:

Upon the extraction of one drop of blood, a microhematocrit was conducted by using heparinized microtubes. The samples were collected at the following times:
Day 5: 08:00 hs, 12:00 hs, 16:00 hs and 20:00 hs.

The collected samples were also subjected to Natremia and Kalemia determination.

Data Analysis: A comparative analysis of the different formulations was performed through descriptive statistics and two-way analysis of variance (ANOVA), followed by Tukey's multiple comparison test to identify differences between different times. These operations were performed with GraphPad Prism 6.0 software.

### RESULTS: Body Mass Recovery:

| | 1M | | 2M | | 3M | | 4M | |
|---|---|---|---|---|---|---|---|---|
| | Media | SEM | Media | SEM | Media | SEM | Media | SEM |
| Day 0 | 137.2 | 3.44093 | 137.8 | 2.332381 | 134.2 | 2.2 | 138.4 | 1.805547 |
| Day 1 | 133.8 | 2.61534 | 134 | 2.387467 | 130.6 | 2.357965 | 143.4 | 2.014944 |
| Day 2 | 128.2 | 2.374868 | 128 | 1.760682 | 123.8 | 3.168596 | 143.8 | 1.714643 |
| Day 3 | 122.8 | 2.596151 | 124.4 | 3.091925 | 120.6 | 3.37046 | 146.2 | 2.853069 |
| Day 4 | 112.8 | 4.465423 | 113.8 | 2.477902 | 110.6 | 3.17175 | 149.6 | 2.088061 |
| Day 5 (8 hours) | 108.6 | 3.613863 | 105 | 2.258318 | 103.4 | 2.158703 | 153.4 | 3.249615 |
| Day 5 (12 hours) | 115.8 | 4.140048 | 110.4 | 1.939072 | 109 | 2.50998 | 153.4 | 2.501999 |
| Day 5 (16 hours) | 124.8 | 4.476605 | 115.6 | 1.122497 | 114 | 2.097618 | 155.8 | 2.557342 |
| Day 5 (20 hours) | 133.4 | 4.905099 | 121.4 | 0.6 | 119.6 | 2.249444 | 156.6 | 2.61916 |
| Day 6 | 137.1 | 5.416641 | 125.4 | 1.939072 | 124.4 | 3.043025 | 157.8 | 2.416609 |
| | 1H | | 2H | | 3H | | 4H | |
| | Media | SEM | Media | SEM | Media | SEM | Media | SEM |
| Day 0 | 118.4 | 0.9273618 | 120.4 | 1.50333 | 120 | 0.9486833 | 119.4 | 2.521904 |
| Day 1 | 112.6 | 1.536229 | 114.6 | 1.469694 | 115 | 1.264911 | 121.2 | 2.709244 |
| Day 2 | 105.6 | 1.28841 | 109.6 | 1.28841 | 107.8 | 2.034699 | 123.8 | 2.61534 |
| Day 3 | 98.8 | 1.714643 | 102 | 1.30384 | 102.8 | 1.907878 | 124.4 | 2.785677 |
| Day 4 | 93.4 | 1.249 | 93.6 | 1.939072 | 96 | 0.8944272 | 126.8 | 2.2 |
| Day 5 (8 hours) | 92.8 | 1.939072 | 89.8 | 1.827567 | 93.2 | 2.437212 | 128.6 | 2.135416 |
| Day 5 (12 hours) | 106.4 | 1.6 | 103 | 2.213594 | 107.8 | 0.9695359 | 127 | 1.949359 |
| Day 5 (16 hours) | 112.6 | 1.4 | 101.6 | 1.860107 | 108.6 | 1.886796 | 126 | 2.073644 |
| Day 5 (20 hours) | 116.6 | 1.886796 | 102.6 | 1.469694 | 113.2 | 1.157584 | 123.4 | 2.420743 |
| Day 6 | 118.6 | 2.420743 | 102.6 | 1.16619 | 110 | 1.449138 | 126.8 | 2.332381 |

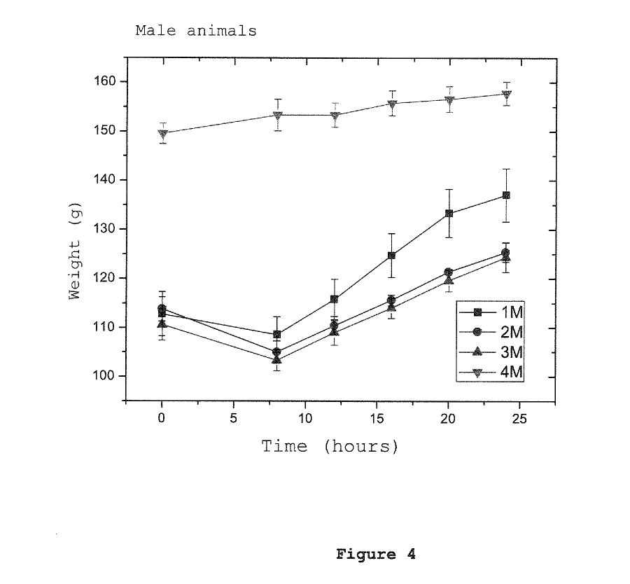

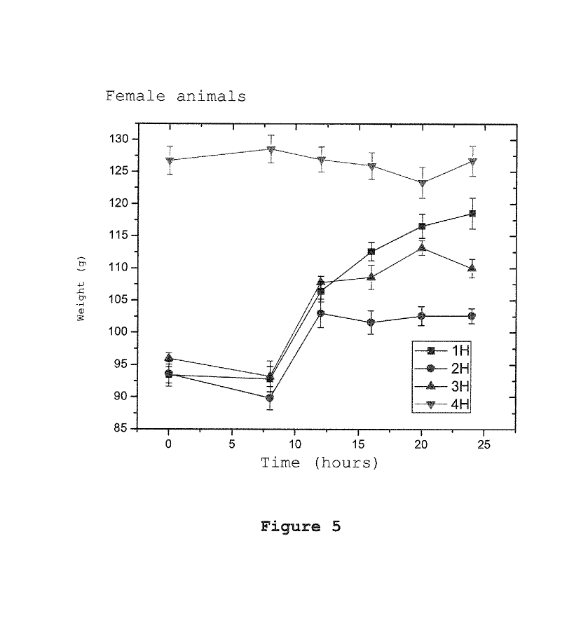

### See FIGS. 4 and 5

### Hematocrit Concentration:

| | 1M | | 2M | | 3M | | 4M | |
|---|---|---|---|---|---|---|---|---|
| | Media | SEM | Media | SEM | Media | SEM | Media | SEM |
| Day 5 8 hours) | 54.6 | 1.029563 | 54 | 0.83666 | 52.4 | 0.4 | 44.4 | 0.6 |
| Day 5 (12 hours) | 48.6 | 0.4 | 50.8 | 0.374166 | 50.6 | 0.6 | 46.2 | 0.583095 |
| Day 5 (16 hours) | 46.6 | 0.4 | 49.2 | 0.374166 | 48.4 | 0.509902 | 45 | 0.316228 |
| Day 5 (20 hours) | 45 | 0.547723 | 48.4 | 0.678233 | 45.2 | 0.860233 | 43 | 0.632456 |
| Day 6 | 44.4 | 1.32665 | 48 | 1.516575 | 46.6 | 2.249444 | 44 | 0.948683 |
| | 1H | | 2H | | 3H | | 4H | |
| | Media | SEM | Media | SEM | Media | SEM | Media | SEM |
| Day 5 (8 hours) | 55.6 | 1.32665 | 54.8 | 0.734847 | 53 | 0.547723 | 46.6 | 0.4 |
| Day 5 (12 hours) | 47.2 | 0.2 | 50.4 | 0.678233 | 50.6 | 0.509902 | 47.2 | 0.583095 |
| Day 5 (16 hours) | 45.8 | 0.2 | 49.6 | 0.6 | 47.8 | 0.2 | 45.4 | 0.979796 |
| Day 5 (20 hours) | 44.4 | 0.509902 | 47.8 | 0.374166 | 45.8 | 1.2 | 44.8 | 0.860233 |
| Day 6 | 43.2 | 0.374166 | 45.4 | 1.630951 | 41.4 | 1.860107 | 42.2 | 1.772004 |

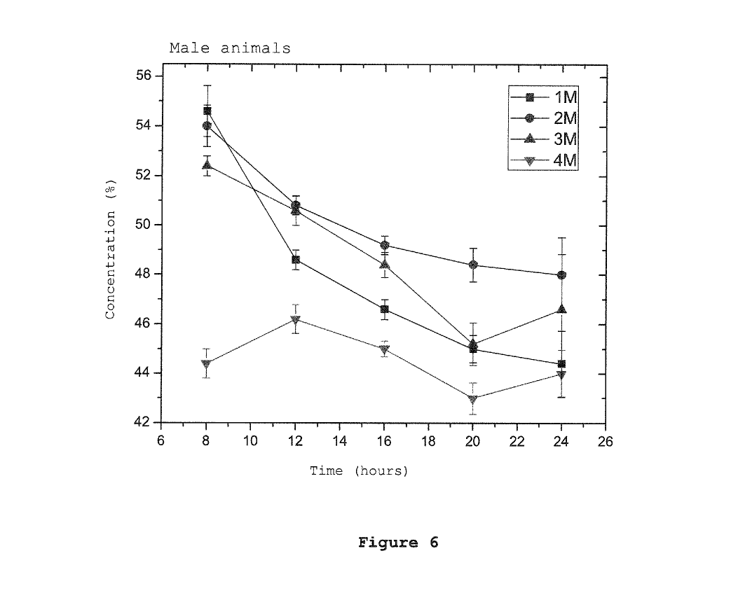

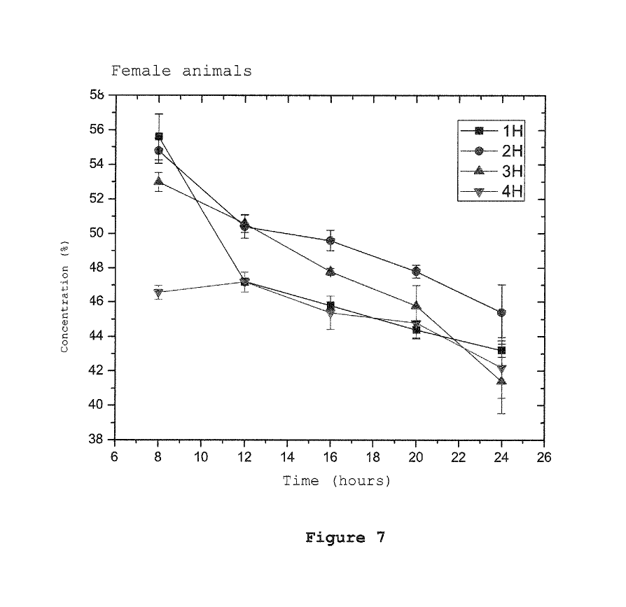

See FIGS. 6 and 7

Natremia (mmol/L):

| | 1M | | 2M | | 3M | | 4M | |
|---|---|---|---|---|---|---|---|---|
| | Media | SEM | Media | SEM | Media | SEM | Media | SEM |
| Day 5 (8 hours) | 200.8 | 3.624914 | 194.6 | 5.1049 | 193 | 4.312772 | 175 | 1.341641 |
| Day 5 (12 hours) | 178.4 | 1.32665 | 191.2 | 1.655295 | 192.4 | 3.893584 | 169.4 | 3.059412 |
| Day 5 (16 hours) | 175.2 | 1.655295 | 190.4 | 1.363818 | 192 | 1.923538 | 173.4 | 2.420743 |
| Day 5 (20 hours) | 175.6 | 1.50333 | 187.2 | 0.7348469 | 189.2 | 1.933908 | 173.2 | 1.496663 |
| Day 6 | 175.2 | 1.593738 | 185.2 | 1.714643 | 187 | 1.48324 | 174.4 | 2.088061 |
| | 1H | | 2H | | 3H | | 4H | |
| | Media | SEM | Media | SEM | Media | SEM | Media | SEM |
| Day 5 (8 hours) | 190.2 | 4.97393 | 194.8 | 3.15278 | 185.6 | 1.8868 | 175.2 | 1.06771 |
| Day 5 (12 hours) | 173.6 | 1.46969 | 192.4 | 2.01494 | 180.2 | 1.06771 | 172.6 | 1.36382 |
| Day 5 (16 hours) | 169.8 | 1.35647 | 189.4 | 2.37907 | 177.2 | 1.15758 | 171.2 | 3.77359 |
| Day 5 (20 hours) | 173.2 | 1.35647 | 188.4 | 1.43527 | 172 | 1.09545 | 174.4 | 2.37907 |
| Day 6 | 171.4 | 3.58608 | 185.2 | 1.88149 | 171.8 | 1.98494 | 174.6 | 1.20831 |

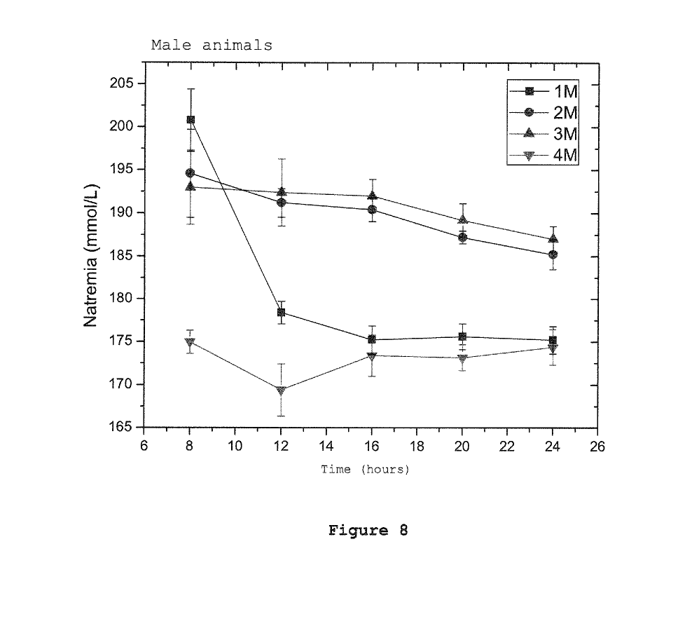

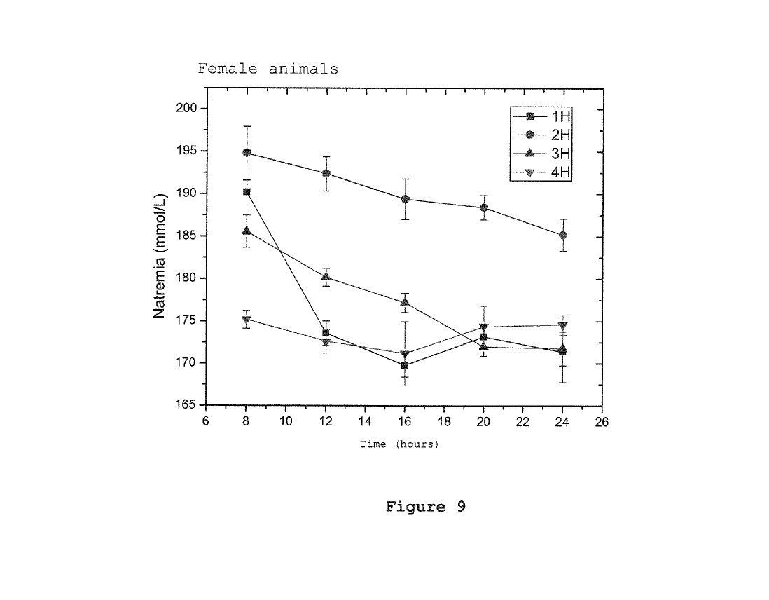

### See FIGS. 8 and 9

Kalemia (mmol/L):

| | 1M | | 2M | | 3M | | 4M | |
|---|---|---|---|---|---|---|---|---|
| | Media | SEM | Media | SEM | Media | SEM | Media | SEM |
| Day 5 (8 hours) | 5.06 | 0.478121 | 4.87 | 0.4895406 | 5.62 | 0.3527038 | 8.48 | 0.2009974 |
| Day 5 (12 hours) | 6.76 | 0.552811 | 5.75 | 0.2792848 | 5.64 | 0.3187475 | 8.4 | 0.2167949 |
| Day 5 (16 hours) | 7.94 | 0.143527 | 6.56 | 0.2466778 | 6.1 | 0.1294218 | 8.139 | 0.1363817 |
| Day 5 (20 hours) | 8.16 | 0.129807 | 7.33 | 0.1299999 | 6.79 | 0.1372953 | 8.36 | 0.1784657 |
| Day 6 | 8.23 | 0.128062 | 7.5 | 0.1695582 | 7.55 | 0.2720294 | 8.4 | 0.0935415 |
| | 1H | | 2H | | 3H | | 4H | |
| | Media | SEM | Media | SEM | Media | SEM | Media | SEM |
| Day 5 (8 hours) | 5.06 | 0.26429 | 5.75 | 0.57619 | 4.9 | 0.54106 | 8.34 | 0.14 |
| Day 5 (12 hours) | 6.52 | 0.69401 | 6.46 | 0.52617 | 5.81 | 0.12288 | 8.42 | 0.08456 |
| Day 5 (16 hours) | 8.58 | 0.09028 | 7.02 | 0.157 | 6.47 | 0.20224 | 8.309 | 0.11662 |
| Day 5 (20 hours) | 8.38 | 0.10794 | 7.41 | 0.17986 | 6.88 | 0.12806 | 8.51 | 0.12787 |
| Day 6 | 8.42 | 0.10794 | 7.51 | 0.11225 | 7.19 | 0.19261 | 8.43 | 0.11023 |

### Conclusions

The osmotic diarrhea model was developed as described in the literature, resulting in significant weight reduction and hematocrit increase due to dehydration.

During dehydration process due to fecal excretion, significant loss of extracellular fluid is produced. Sodium concentration in this fluid is about 30 times higher compared to potassium concentration. During the process of fluid loss, significant loss of solutes is also observed, including sodium and potassium ions, responsible for regulating liquid restitution in the body. However, the percentage of potassium loss is higher than that corresponding to sodium. In addition to liquid reduction, this makes the initial dehydration condition show plasma sodium concentration values higher than those belonging to animals that did not experienced dehydration, and plasma potassium concentration values lower than those of non-dehydrated animals.

The results obtained from the body mass analysis indicated that, as a consequence of diarrhea induction, all the experimental groups by the time treatment was initiated had lost about 20% of their body mass. Thereafter, comparative results showed a significant difference between weight regain in the group treated with the formulation of Example 6 of the present invention and the group treated with Pedialyte®. Both in male and female rats after 24 hours of treatment, the formulation of the present invention induced recovery of average body mass in the experimental group.

Hematocrit is the percent of the total volume of whole blood that is composed of red blood cells. Hematocrit loss during dehydration due to fecal excretion is negligible. This implies that the reduction of plasma extracellular fluid makes hematocrit increase.

On the basis of the condition at the time treatment was initiated, it is possible to see that all the induced groups have a hematocrit level higher than 50%, where all normal values always range from 40% to 50%. The treatment results indicate that recovery in the hematocrit level in the group treated with the formulation of the present invention was significantly faster than that achieved by Pedialyte®. In male rats, normal level was achieved after 24 hours of treatment, whereas in female rats the action was much more effective, the normal level being recovered after 8 hours of treatment.

Natremia and Kalemia analyses are highly influenced by extracellular fluid recovery. Reduction in sodium concentration in all the experimental groups does not mean there is cation loss, but a reduction in cation concentration. This means the body absorbs sodium and recovers a higher liquid percentage; thus, its concentration diminishes. The experimental results revealed that both in male and female rats, the recovery rate of normal sodium and potassium levels was significantly higher for the formulation of the present invention compared to Pedialyte®.

It should be noted that the term “osmolality” refers to moles per kilogram and the term “osmolarity” refers to moles per liter. They are different terms, but throughout this description, they could be used interchangeably due to low density values that enhance the low impact on the conversion of osmolality to osmolarity of the liposomal rehydration salt formulation as described.

It is also possible to use a modified manufacturing process than that described above by not creating an ethanol phase, and instead, creating a concentrated phase that already has the liposomes. That concentrated phase is then mixed as an ingredient with the end product. It has also been found that the inclusion level of salts can be increased to as high as 70% in some conditions versus inclusion levels such as 54% or 56%. In an aspect of manufacturing, the concentrated phase of liposomes has been developed in a ratio of 1:20 and the end product profile has been upgraded, replacing HFCS such as high fructose corn syrup, with glucose and Stevia and other sugars while also using natural flavors and colors. A variety of ranges are identified above for the liposomal rehydration salt formulation. A specific composition/formulation specification explained below shows the different specifications for sodium, potassium, chloride, citrate, glucose, carbohydrates and calories. This current example is also known as Speedlyte® and is later compared to another non-liposome formulation sold by Abbot as Pedialyte® and compared to WHO (World Health Organization) 2002 recommendations.

### Example Composition/Formulation Specifications

| | | | | |
|---|---|---|---|---|
| Sodium | 1,035 | mg/L | 45 | mEq/L |
| Potassium | 782 | mg/L | 20 | mEq/L |
| Chloride | 1,380 | mg/L | 39 | mEq/L |
| Citrate | 748 | mg/L | 8.7 | mEq/L |
| Glucose | 13.50 | g/L | 75 | mEq/L |
| Total Carbohydrates | 25.5 | grams | | |

| | |
|---|---|
| Calories | 90 |

In this example, based on liposomeelectrolytes, the actual composition/formulation osmolarity should be taken as 125.8 mmol/L based on a current theoretical osmolarity of 188, with 54% of the electrolytes being encapsulated.

These example values in the table above can vary from 5%, 10%, 15%, 20% and 25% above and below these values, although the greater percentage difference from the listed values is less desirable. Based on the liposome electrolytes, the actual composition/formulation osmolarity should be taken as 125.8 mmol/l based on a current theoretical osmolarity of 188, with 54% of the electrolytes being encapsulated. The osmolarity analysis (meq/L) is referred to as milliequivalents of solute per liter of solvent and is the amount of substance that reacts or is equivalent to another amount of substance. Of course, the equivalent is the amount of a substance needed to react with or supply one or more hydrogen ions in an acid-base reaction or react with or supply one or more of electrons in a redox reaction.

### Osmolarity Analysis (meq/L)

| | | | Speedlyte ® |
|---|---|---|---|
| | WHO 2002 | Pedialyte ® | (conventional/liposomed) |
| Sodium | 75 | 45 | 45/20.2 |
| Chloride | 65 | 35 | 39/17.5 |
| Potassium | 29 | 20 | 20/9 |
| Citrate | 10 | 10 | 9/4 |
| Glucose | 75 | 139 | 75/75 |
| Total | 245 | 249 | 188/125.8 |
| Osmolarity | | | |

Values can vary from 5%, 10%, 15%, 20% and 25% for the current formulation.

In the human body, the amount of a substance and equivalence is a very small magnitude and it is routinely described in terms of milliequivalents (meq) as the measure having been multiplied by 1,000. The osmolarity analysis comparing the World Health Organization 2002 standards (WHO 2002) with the formulation manufactured by Abbot as Pedialyte® and an example of the current formulation also referred to as Speedlyte® are illustrated above and compare the example values. The Pedialyte® composition is a non-liposomal formulation that includes a number ingredients as an oral fluid and electrolyte replacement. As shown in the table, it is evident that its osmolarity is much higher than the osmolarity of the current formulation. One differentiating factor in the marketplace and for efficacy is the lower osmolarity of the current formulation and also has beneficial aspects in its higher absorption results. Also, the electrolytes and liposomes serve as either a maintaining formula or a rehydration formula. The percentage deviation ranges described above are applicable and the current formulation as shown in the tables has 45 mEq/L of sodium and 20 mEq/L of potassium from 2.28 g/L of sodium chloride plus 2.04 g/L of potassium citrate and 0.5 g/L of sodium citrate. The more important electrolyte is sodium and that can range from 12 mEq/L to as high as 90 mEq/L. Other intermediate ranges have been found acceptable such as 20 to 70, 30 to 60, 35 to 55, and 40 to 50 mEq/L for the sodium. The potassium electrolytes can also vary based on the percentages described above and in one example is about 15 mEq/L to 25 mEq/L. Phospholipids help create the liposomes and the liposome concentrations and the range identified above is 1 to 60 g/L with other ranges as defined in the examples with 1 to 30, 1 to 40, or 1 to 50 g/L with the specific concentrations of 30, 40 and 50. The example formulation as described in the tables above has 2.5 g/L of phospholipids and that value can range from about 1 to 5 g/L, 1 to 10 g/L, in a preferred example, 5 to 10 g/L and up to 30, 40, 50 or 60 g/L.

The amount and type of carbohydrates may vary and in one example, the carbohydrates are at a concentration of up to 6 g/L and up to 30 g/L. The current formulation as described in the tables has as carbohydrates 13.5 g/L of glucose and 11.5 g/L of sugar, and in an example may range from about 8.0 g/L to about 15.0 g/L of at least one additional sugar. The level of carbohydrates may be increased to as high as 70 g/L to appeal to a more mainstream product and it may be lowered to almost 0 g/L to appeal to diabetics and the elderly. Those ranges of glucose and sugar can vary from their mid-range value at 5%, 10%, 15%, 20%, and 25% differences above and below.

The size of the liposomes are important for absorption as discussed above and can vary as noted above and is preferred about at 225 to 450 nm, but can range in an example from 200 to 500 nm. Although some available commercial products have smaller sized liposomes as alleged by their manufacturers, such as 100 mm, it has been determined their level of inclusion volume is low. Other oral liposome products, for example, using vitamin C, may have liposome sizes greater than 500 nm.

In the example 7 described above, natural flavors are concentrated at about 1.5 g/L. Natural flavorings and masking flavors may be at a concentration of up to 5 g/L. The example formulation shown in the charts above has about 3.12 g/L flavorings and these values could vary from 5%, 10%, 15%, 25%, or 25% above and below those values. One example range is about 1.0 g/L to about 3.5 g/L. Stevia may be used as a natural sweetener and in example, the Stevia in the examples above is indicated at a concentration of about 0.1 to 0.2 g/L, and in the example shown in the tables, is currently about 0.15 g/L, but it is possible to go as high as 0.22 in one example. One example range is about 1.0 g/L to about 3.5 g/L.

The liposomal rehydration salt formulation not only maintains hydration, but also operates to rehydrate those persons that are dehydrated, in some cases serious. For example, it is possible to rehydrate and avoid the intravenous (IV) fluid delivery necessary in some cases because the formulation may be orally administered to humans suffering from dehydration caused by various factors as indicated below or suffering a moderate to severe dehydration with the need for IV fluids. The formulation also will allow fluid maintenance and avoid use of IV fluids by preventing severe dehydration while maintaining body electrolytes and fluids. The formulation may be used for rehydration due to stomach bugs in children and adults and hydration to prevent hangover or rehydration while in a hangover episode. The liposomal rehydration salt formulation may prevent or avoid the need for parenteral hydration, corresponding to fluids that are injected subcutaneously such as parenteral glucose or saline.

The liposomal rehydration salt formulation may be used for hydration or rehydration for pregnant or breast-feeding women and for persons that need hydration or rehydration due to exercise (sport), outdoor activities, extreme weather or high-altitude conditions, skin burns, flying, hangover episodes, diarrhea, vomiting, high fever, stomach bugs or other types of gastritis, norovirus, rotavirus, and other types of bacteria and infections. Hikers that are climbing steep hills or cliffs may also find the liposomal rehydration salt formulation advantageous to help in maintaining body electrolytes and in hydration or rehydration.

The liposomal rehydration salt formulation may also be used for hydration or rehydration to different populations, including patients with parenteral and enteral nutrition, to reduce the volume and consequently the time of intravenous (IV) treatments. It may be used with celiac patients, particularly during an episode such as when a person may inadvertently eat protein to which they may be allergic such as found in wheat, rye, and barley and their body mounts an immune response. The symptoms may include abdominal bloating, pain, gas, diarrhea, pale stools and weight loss. The liposomal rehydration salt formulation may also be used with elderly patients, pediatric patients, pregnant and breast-feeding patients, and diabetic patients, particularly those under a SGLT2 inhibitor treatment corresponding to a class of prescription medications that inhibit sodium-glucose transport protein 2 and that react to reduce blood to glucose levels. Thus, the liposomal rehydration salt formulation can be especially effective for those type of patients. Such class of medications are sometimes referred to as gliflozin drugs that inhibit the reabsorption of glucose in the kidney and therefore lower the blood sugar, sometimes too much.

The liposomal rehydration salt formulation as described may also be used for those that suffer from intestinal failure, including the Short Bowel Syndrome, also called short gut, as a malabsorption disorder caused by the lack of a functioning small intestine. Diarrhea is a typical symptom that sometimes results in dehydration, malnutrition and weight loss.

Other populations that will benefit from the liposomal rehydration salt formulation include those that suffer from Cycling Vomiting Syndrome (CVS) with the sudden, repeated attacks as episodes of severe nausea, vomiting and physical exhaustion that could last a few hours to several days. Other persons suffering from gastroparesis as, for example, delayed gastric emptying with paresis of the stomach and food often remaining in the stomach for an abnormally long time, which may cause chronic nausea and vomiting in some cases with erratic blood glucose levels. Those suffering from Postural Orthostatic Tachycardia Syndrome (POTS) characterized when too little blood returns to the heart when moving from a lying down to a standing up position corresponding to orthostatic intolerance may benefit from the formulation. Those suffering from ulcerative colitis and colon cancer could use the liposomal rehydration salt formulation to benefit them since often they become dehydrated.

Also, those having dysphagia and difficulty swallowing could benefit as well as those with Sjogren's syndrome, corresponding to a systemic autoimmune disease that may include dry eyes and dry mouth and often is accompanied by rheumatoid arthritis and lupus. Those with lupus or similar immune disorders may also benefit from the liposomal rehydration salt formulation. Especially beneficial would be those suffering from Crohn's Disease and lupus with typical abdominal cramping and pain as part of a chronic Inflammatory Bowel Disease (IBD) and inflammation of the digestive or gastrointestinal (GI) tract. Crohn's Disease is usually limited to the end of the small intestine, as compared to ulcerative colitis, which is usually limited to the large intestine such as the colon and rectum. The liposomal rehydration salt formulation is beneficial for sufferers of either disorder. Those having kidney disease with dangerous levels of fluid, electrolytes and waste build up will benefit from the use of the liposomal rehydration salt formulation.

Those suffering from HIV are especially benefitted since often treatment for HIV and AIDS causes vomiting and diarrhea. Diarrhea is a typical side effect that accompanies use of HIV medications used for AIDS treatment. Often this includes nausea and headache with some fever, accompanied by vomiting and diarrhea. The liposomal rehydration salt formulation is especially beneficial in this type of treatment not only to help maintain electrolytes, but for rehydration. Some antiretroviral drugs for AIDS may increase blood sugar and diabetes and the liposomal rehydration salt formulation is beneficial.

Those with the Inflammatory Bowel Disease (IBD) such as Crohn's Disease and ulcerative colitis would benefit as well as those with an ostomy, i.e., a stoma as a surgically created opening between the intestines and abdominal wall, and thus typically requiring a bag or pouch. This may can cause glucose levels and other electrolyte changes in the body, especially in the blood and intestines. Those suffering from microvillus inclusion disease also termed Davidson's Disease also suffer from chronic, intractable diarrhea causing metabolic acidosis and severe dehydration and thus would benefit from use of the liposomal rehydration salt formulation. Those suffering from cystic fibrosis (CF) would benefit from its use. Although CF is a genetic order affecting the lungs, it may also affect the pancreas, liver, kidneys and intestines causing difficulty in breathing. It can also cause fatty stool. The liposomal rehydration salt formulation can be used in many different types of cancer treatment and especially those suffering from HIV symptoms.

The liposomal rehydration salt formulation has superior taste, higher absorption, less intake and lower sugar than other drinks and formulations both using and not using liposomal technology. Testimonies have stated that individuals feel better hydrated after taking the disclosed liposomal rehydration salt formulation and have fewer headaches and less need for IV fluids. Taken regularly, it especially may provide those suffering from gastroparesis, Crohn's Disease, ulcerative colitis, POTS, SBS, and colorectal cancer to reduce IV hydration and hospital visits, increase energy, reduce palpitations, feel less thirst, experience fewer headaches, eliminate cramps and reduce dizziness. It is found that the liposomal rehydration salt formulation hydrates in one-third of the time and requires only one-third of the intake as compared to many other hydration drinks. The current formulation also uses 46% less sugar than many commercially available hydration drinks. The current formulation aids those that live with the risk of dehydration and it is better than water since water is a very poor hydrator for moderate and severe situations and may cause loss of fluid greater than the amount of fluid consumed, thus leading to electrolyte imbalance. Even coconut water often may not contain enough electrolytes for maintaining proper hydration in severe and chronic dehydration situations. Usually, sports drinks do not contain enough electrolytes because they are designed for mild dehydration situations. The electrolyte powders and tablets that are commonly available present absorption limitations and bad taste. Many commercially available oral rehydration solutions (ORS) are also old technology as compared to the current liposomal rehydration salt formulation that uses nano-electrolytes for higher fluid absorption, less intake requirements and better taste. The formulation can be diluted with water, juices and other drinks, depending on the electrolyte level required.

It is possible in some cases to add small amounts of other functional ingredients as part of the liposome technology, including vaccines, drugs, amino acids, mineral salts, vitamins, nutraceuticals, probiotics, prebiotics, and other flavors, including nutritive and non-nutritive sweeteners. The amounts would vary of course depending on end uses.

There are also many disorders from alcohol abuse and the liposomal rehydration salt as described in an example may incorporate a composition such as oleoylethanololamide (OEA) and/or related compounds as described in patent application PCT/ES2017/070225 filed on Apr. 11, 2017, and entitled, “Compositions for the Prevention and/or Treatment of Alcohol Use Disorders,” based on Spanish patent application P201630447 filed on Apr. 11, 2016, the disclosures which are hereby incorporated by reference in their entirety.

An example liposome formulation could include the liposome formulation as described above that adds OEA to the salts and encapsulates some or most of the OEA. Many medications used to treat alcohol related disorders have serious side effects, and it is necessary to have the joint administration of several drugs for the treatment of the symptoms and pathologies that accompany the addition of alcohol. It would therefore be good to develop treatment alternatives that have fewer side effects and to act on as many of the harmful effects of alcohol in the organism, as well as preventive treatments of the damage caused by the consumption of alcohol at different levels and in different organs. Thus, the benefits of OEA are incorporated with rehydration formulation described above.

A liposomal formulation includes phospholipids at a concentration of 1 g/L to 60 g/L, salts, water, and a percentage inclusion ratio of salts (salts retained within total salts/liposomes) of at least 40%, and a compound of general formula (I)

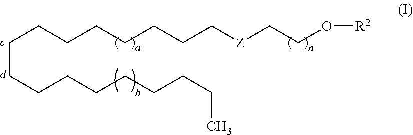

or any of its pharmaceutically acceptable salts, esters, tautomers, solvates and hydrates, or any combination thereof, where:

n is an integer ranging from 0 to 5;

a and b are determined by the following formula: 0≤(a+b)≤4; and

Z is a group selected from —C(O)N(R0)-; —(R0)NC(O)—; —(O)CO—; OR; NR0; And S, and wherein R0 and R2 are independently selected from the group consisting of substituted or unsubstituted alkyl, hydrogen, substituted or unsubstituted (C1-C6) alkyl, substituted or unsubstituted (C1-C6) acyl, and aryl, and wherein up to eight hydrogen atoms of the compound may be substituted by methyl or a double bond, and the molecular bridge between c and d may be saturated or unsaturated. The formulation has an osmolality lower than 190 mmol/L.

This compound may be formed as oleoylethanolamide. In the formulation, a percentage inclusion ratio of salts (salts retained within total salts/liposomes) may be at least 40% or 50% or 54% or higher. The phospholipids may be selected from the group consisting of phosphatidylcholines (PCs), phosphatidylserines (PSs), phosphatidylethanolamines (PEs), phosphatidylglycerols (PGs), phosphatidylinositols (PIs), phosphatidic acids (PAs), and mixtures thereof.

In yet another example, an antioxidant is selected from the group consisting of phytosterol, tocopherol, and mixtures thereof. The formulation has an osmolality lower than 190 mmol/L, and with the inclusion of salts, the actual osmolality is lower than 130 mmol/L. The liposomes may comprise a particle diameter ranging from 200 to 500 nm and as noted above, about 225 nm to 450 nm. The formulation in an example is an oral administration infusion for individuals that have an alcohol related disorder.

In yet another example, a method for preparing a liposomal formulation that includes phospholipids at a concentration of 1 to 60 g/l, salts, water, and a compound of general formula (I) as an example oleoylethanolamide, and an inclusion ratio as noted before such as of at least 40% and may include:

a. preparing an aqueous phase (AP) or buffer comprising sodium chloride, potassium citrate, sodium citrate dissolved in distilled water (the compound of general formula (I) as an example as oleoylethanolamide is added);

b. separately preparing an ethanol phase (EP) by dissolving said phospholipid at a concentration of 0.1 to 6% (W/V) and optionally an antioxidant at a concentration of 0.2 to 0.5% (W/V), in alcohol (more compound of general formula (I) as example oleoylethanolamide may be added);

c. inducing formation of liposomes by injecting said EP into said AP at room temperature and while stirring and wherein the compound of general formula (I) is:

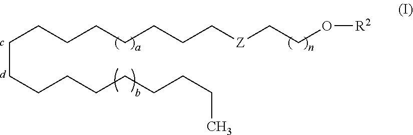

or any of its pharmaceutically acceptable salts, esters, tautomers, solvates and hydrates, or any combination thereof; where:

n is an integer ranging from 0 to 5;

a and b are determined by the following formula: 0≤(a+b)≤4; and

Z is a group selected from —C(O)N(R0)-; —(R0)NC (O)—; —(O)CO—; OR; NR0; And S, and wherein R0 and R2 are independently selected from the group consisting of substituted or unsubstituted alkyl, hydrogen, substituted or unsubstituted (C1-C6) alkyl, substituted or unsubstituted (C1-C6) acyl, and aryl, and wherein up to eight hydrogen atoms of the compound may be substituted by methyl or a double bond, and the molecular bridge between c and d may be saturated or unsaturated;

d. subjecting the liposomal solution obtained in step c to a tangential ultrafiltration (TUF) concentration process, removing the buffer and maintaining the liposomes and their contents, thus reducing the volume at least by 10-fold; and

e. subjecting the liposomal solution obtained in step d to a tangential ultrafiltration (TUF) concentration process, wherein ethanol is removed and the buffer is replaced by a saline solution, maintaining the liposomes and the contents therein.

Although a specific manufacturing process is described above relative to the salts and also the added OEA in an example, other manufacturing processes and methods may be used, including liposome preparation such as by sonication, detergent dialysis, ethanol injection or dilution, French press extrusion, ether infusion and reverse phase evaporation. Though liposomes have multiple bilayers that are also known as multilamellar lipid vesicles and also are useful for time release drugs because the fluids and ingredients entrapped between the layers are released as each membrane degrades. There may also be single bilayers known as unilamellar lipid vesicles and may be small or large in dimension. The liposomes could be partitioned into different lipid bilayers or include an aqueous interior space. It is possible to prepare lipid vesicles using ethanol injection type processes, including use of a static mixer to provide a turbulent environment such as a Reynolds number greater than 2,000 and any agents may be loaded after vesical formation.

The inclusion ratio of salts and the preparation and amount of salts within the liposome rehydration salt formulation as described above and may include the compound one as described below and OEA. The OEA may be in the range of about 0.1 to 200 mg per daily dosage serving of the formulation, which could in some cases be a 20 ounce serving of an oral electrolyte solution with the encapsulated nano-electrolytes and OEA, but could also in one example range from about 1 to 50 mg per serving of the formulation, and in other examples, 1 to 40 mg, 1 to 30 mg, 1 to 20 mg, or in one preferred range, about 5 to 30 mg, and in an yet another example, as high as 60 or 70 or 80 mg. These amounts can vary between 5, 10, 15, 20, and 25% in value above and below. The OEA may have a percentage inclusion ratio of total OEA/liposomes or other compounds such as compound one of at least about 40%. It may be higher also depending on how much OEA is included within liposomes with salts or without salts.

There now follows some background information on the alcohol related disorders and how OEA and similar counterparts may be used to aid in treating the disorders and especially with the use of liposomes as described above.

The ICD-10 (acronym of the International Classification of Diseases, tenth version) classifies with the code F10 the mental and behavioral disorders due to the use or consumption of alcohol. Code F10 in turn comprises the following subsections:

F10.0. Acute intoxication.
F10.1. Harmful consumption.
F10.2. Syndrome of dependence.
F10.3. Abstinence syndrome.
F10.4. Abstinence syndrome with delirium.
F10.5. Psychotic disorder.
F10.6. Amnestic syndrome.
F10.7. Residual and late psychotic disorder.

### F10.8. Other Mental Disorders Induced by Alcohol.

F10.9. Unspecified mental or behavior disorder.

The clinical criteria for each diagnosis are included in the corresponding section within the chapter. Harmful consumption (F10.1) means the one that is affecting the physical or mental health, without fulfilling the criteria of dependence or any other of those indicated within F10. All the most recent epidemiological studies on alcohol abuse and dependence in Western countries show that harmful consumption is a growing problem, increasingly serious, and increasingly characteristic of young people, between 15 and 30 years of age. Most studies estimate that the lifetime risk of alcohol dependence is currently about 9-10% for men and 3-5% for women. The same risk for harmful consumption would be almost double. Alcohol or alcohol intoxication is a pattern of alcohol consumption common among some alcoholics or alcohol dependent. It has been proposed that brain damage and alcohol-induced neurodegeneration is a direct consequence of binge drinking episodes, and there is compelling evidence that neuro-inflammation induced by these may contribute to the neurotoxic effects of the drug. Four-day models of alcohol intoxication (12 intra-gastric doses), the so-called Majchrowicz intoxication model, and minor modifications thereof, have been widely used to describe neuro-inflammation and neurodegenerative properties of alcoholic intoxication.

For example, alcohol intoxication causes the activation of microglia, increases the binding activity of nuclear factor-kappa B (NF kappa B) to DNA, increases the expression of cyclooxygenase-2 (COX-2), and induces brain injury and neurodegeneration in the cerebral cortex and hippocampus associated with cognitive deficits. In addition, the cited alcohol intoxication model induces high-prolonged blood ethanol levels (BEL) similar to those documented in chronic alcoholics.

Chronic administrations of ethanol increase inflammatory mediators related to NF-kB and activate the signaling pathways of Toll-like receptors (TLRs) 4 and 3, inducing apoptosis, brain injury and neurodegeneration. Neuroimmune activation induced by chronic alcohol administration may involve the warning signaling molecule HMGB1 (High Mobility Group Box 1), which binds to TLR4.

An increase in HMGB1 expression has been observed in the orbitofrontal cortex of the postmortem alcoholics and their levels correlated with lifetime alcohol consumption. It has also been documented in humans alcohol-dependent increases in peripheral pro-inflammatory cytokines such as tumor necrosis factor alpha (TNF-α) and interleukin-1 beta (IL-1β), activation of microglia and increased brain expression of monocyte chemoattractant protein 1 (MCP-1). Inflammation was also correlated with depressive symptoms and craving for alcohol.

The endocannabinoid system has been studied for years for its anti-inflammatory and homeostatic properties. A structural analogue of the endocannabinoid anandamide belonging to the N-acylethanolamine family, the lipid mediator oleoylethanololamide (OEA), has emerged as an interesting bioactive molecule with anti-inflammatory and neuroprotective actions in the brain. OEA was first discovered as a satiety factor with no activity in traditional cannabinoid receptors, and there is increasing evidence that it mediates a variety of different actions through the activation of the peroxisome proliferator-activated receptor, alpha subtype (PPAR-A). Recent studies in animal models indicate that OEA may have putative neuroprotective properties against central nervous system (CNS) disorders such as stroke, Parkinson's disease, depression or addiction.

Alcohol, specifically ethanol, is a potent psychoactive drug with a high number of side effects that can seriously affect our body. The effects of alcohol are very diverse in the medium and long term, and act on multiple organs and systems as detailed below. In the brain and nervous system. It gradually affects the brain functions, first to the emotions (sudden changes of humor), the processes of thought and the judgment. Continued alcohol intake disrupts motor control, causing dysarthria, nervous dullness and loss of balance. It also alters the action of neurotransmitters, and modifies their structure and function. This produces multiple effects: decreased alertness, delayed reflexes, changes in vision, loss of muscle coordination, tremors, and hallucinations. Decreases self-control, affects memory, ability to concentrate and motor functions. Decreased vitamin B1 produced by chronic alcohol consumption can lead to Wernicke-Korsakoff disease, which causes cognitive deficits and alterations in the person's feelings, thoughts and memory. Those affected confuse reality with their inventions. It produces sleep disorders or progressive loss of memory and other mental abilities.

In advanced stages, it produces serious mental alterations and irreversible brain damage, the so-called alcoholic dementia. Periods of amnesia, with profound alteration of memory and awareness of different duration (minutes, hours or even days). It also increases cardiac activity and very moderate consumption improves circulation, but a higher dose causes damage. In high doses, it increases blood pressure (hypertension) and produces damage to the heart muscle due to its toxic effects. It also weakens the cardiac musculature and therefore the ability to pump blood and produces peripheral vasodilatation, which causes redness and an increase in the surface temperature of the skin.

In the digestive system, the alcohol has an effect and the gastric discomfort is due to erosions in the mucous membranes produced by ethanol. The heartburn will be greater if different drinks or mixed drinks have been mixed, since the gastric irritation will be due to all the components drunk. It increases the production of gastric acid that generates irritation and inflammation in the walls of the stomach so that, in the long term, ulcers, bleeding and perforations of the gastric wall can appear. Stomach cancer has been linked to alcohol abuse. It also causes cancer of the larynx, esophagus, pancreas, and in some cases of bladder. It causes esophagitis, an inflammation of the esophagus, bleeding esophageal varices and Mallory-Weiss tears and may produce acute pancreatitis, a severe inflammatory disease of the pancreas, which is life threatening. It can cause chronic pancreatitis, which is characterized by intense permanent pain. Other possible alterations may be type 2 diabetes and peritonitis.

The liver is the organ responsible for metabolizing alcohol, which is transformed by liver enzymes first into acetaldehyde and then into acetate and other compounds. This process is slow and harmless since acetaldehyde depolarizes proteins, oxidizes lipids, consumes B vitamins and damages tissues. Hepatic cell irritation may lead to alcoholic hepatitis due to cell destruction and tissue inflammation. Over time, the liver evolves (fatty liver or steatosis) to adapt to metabolic overload, being able to reach hepatitis and later to liver cirrhosis, a product of cell death and degeneration of the organ. This serious disease can eventually degenerate into liver cancer and lead to death. Other signs of hepatic impairment are jaundice, a yellowing of the skin and sclera, and edema, accumulation of fluid in the extremities. It also alters the function of the kidney, reducing the antidiuretic hormone levels, causing dehydration and taking water from other organs such as the brain, which generates headache. Alcohol provides plenty of calories (7 kcal per gram of alcohol) with little nutritional value. It does not nourish but it eliminates the appetite, it substitutes to other more complete foods and in the long run it can generate malnutrition. This is aggravated as it inhibits the absorption of some vitamins and minerals.

In the blood, alcohol may inhibit the production of white and red blood cells and without enough red blood cells to transport oxygen, megaloblastic anemia ensues. In the immune and reproductive systems, the lack of white blood cells causes a failure in the immune system, increasing the risk of bacterial and viral infections. It decreases libido and sexual activity and can cause infertility and erectile dysfunction.

In addition, alcoholics often present with other psychiatric syndromes, especially anxiety and depression, which are often induced disorders or aggravated by alcohol consumption itself. Depression is a pathology that is frequently associated with alcoholism (36% of alcoholic patients suffer from depression at the same time). This association is more frequent in women than in men, and has a highly negative effect on the evolution of patients Alcoholics by increasing their disease relapse and overshadowing the forecast of it.

Currently, drugs used for the treatment of alcoholism are often directed to the treatment of addiction, and sometimes other associated diseases such as depression and anxiety. Many of these drugs have side effects.

For depression and addition to alcoholism, (bromazepam plus sulpiride), amitriptyline, fluoxetine hydrochloride have been used and for anxiety, (bromazepam plus sulpiride), alprazolam, amitriptyline, chlordiazepoxide hydrochloride, chlorazepate dipotassium, diazepam, hydroxyzine, hydroxyzine hydrochloride, meprobamate, thiapride, Ulcipep (chlordiazepoxide plus clidinium bromide) are also indicated.

There now follows a description of the OEA and related components and compounds that may be encapsulated within the liposome formulation as a liposome rehydration salt formulation for alcohol treatment and preferably includes some salts as described before with the additional OEA. However, OEA could be encapsulated alone, if during preparation, the OEA is alone without the salts. However, use of salts as disclosed is preferred. Details of the testing of the OEA are now described.

The temporal profile of neuro-inflammation in rats exposed to excessive intragastric ethanol administration (3 times/day×4 days) and the anti-inflammatory/neuroprotective properties of oleoylethanolamide (OEA) were tested. It has been found that the OEA, administered pretreatment during alcohol intoxication, exerts antidepressant-like effects during acute withdrawal. Taken together, the results demonstrate a beneficial OEA profile as a potent anti-inflammatory, antioxidant, neuroprotective, and antidepressant compound to treat alcohol intoxication and disorders associated to alcohol consumption.

It is noted that the results detailed could be generalized to ethanolamides of a fatty acid, therefore, the composition also relates to the use of an ethanolamide of a fatty acid also possible in the liposomal rehydration salt formulation, but also in the manufacture of a medicament, drug, dietary supplement, medical food or nutraceutical for the prevention, relief, amelioration and/or treatment of alcohol use disorders or consumption, of a disease or disorder caused by the ingestion of alcohol, veisalgia or hangover.

Therefore, a first aspect relates to the use of a compound of general formula (I):

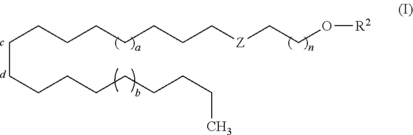

Where n is an integer ranging from 0 to 5; The addition of a plus b situates in the 0 to 4 range; Z is a group selected from —C(O)N(R0)-; —(R0)NC(O)—; —(O)CO—; OR; NR0; and S, and wherein R0 and R2 are independently selected from the group consisting of substituted or unsubstituted alkyl, hydrogen, substituted or unsubstituted (C1-C6) alkyl, substituted or unsubstituted (C1-C6) acyl, and aryl, and wherein up to eight hydrogen atoms of the compound may be substituted by methyl or a double bond, and the molecular bridge between c and d may be saturated or unsaturated; or any of the pharmaceutically acceptable salts, esters, tautomers, solvates and hydrates thereof or any combination thereof to be incorporated with the liposomal formulation as described above. It is also useful in the manufacture of a medicament, drug, dietary supplement or nutraceutical for the prevention, amelioration and/or treatment of conditions for use or consumption of alcohol in a mammal.

Alternatively, it relates to the compound or any salts thereof, preferably any pharmaceutically acceptable salt, esters, tautomers, polymorphs, hydrates, or an isomer, prodrugs, derivatives, solvates or the like, or any combination thereof, for use in the prevention, alleviation, amelioration and/or treatment of alcohol use disorders in mammals when used in the liposome technology described above.

In another embodiment, the compound has the formula (II):

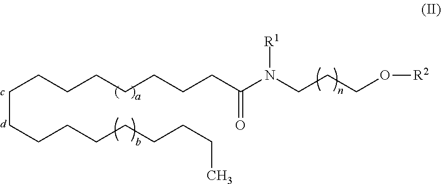

Where m is an integer ranging from 0 to 4; the addition of a plus b situates in the 0 to 3 range; R1 and R2 are independent members which are selected from the group consisting of a substituted or unsubstituted (C1-C6) alkyl, substituted or unsubstituted (C1-C6) acyl, and wherein up to eight hydrogen atoms of the compound may be substituted by a methyl or a double bond, and the molecular bridge between c and d may be saturated or unsaturated; or any of its salts, preferably a pharmaceutically acceptable salt, esters, tautomers, polymorphs, pharmaceutically acceptable hydrates, or an isomer, prodrugs, derivatives, solvates or the like, or any combination thereof.

In another embodiment, a=1 and b=1. In another embodiment, n=0-1. In another embodiment, R1 and R2 are hydrogens (H). In another embodiment, the bridge between carbon c and carbon d is a double bond. In another embodiment, the compound of formula (I) is an acylethanolamide. The acetylethanolamide is selected from the group consisting of oleoylethanolamide (OEA), palmitoylethanolamide (PEA), stearoylethanolamide (SEA) or any combination thereof. Even more preferably, the acylethanolamide is oleoylethanolamide (OEA) of formula (III).

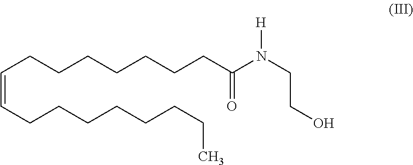 [SMILES: CCCCCCCC/C=C\CCCCCCCC(=O)NCCO]

The term “almitoylethanolamide” refers to the compound whose structure is:

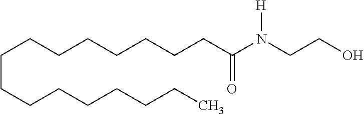 [SMILES: CCCCCCCCCCCCCCCCC(=O)NCCO]

In other aspects, the formulation relates to fatty acid ethanolamide compounds, homologues, analogs; and their pharmaceutical compositions, as well as to uses. In other embodiments, the fatty acid alkanolamide fatty acid residue or ethanolamide, homologous or analogous compound may be saturated or unsaturated, and if unsaturated may be monounsaturated or polyunsaturated.

In some embodiments, the fatty acid moiety of the acid alkanolamide compound, fatty acid, homologue or analogue is a fatty acid selected from the group consisting of oleic acid, palmitic acid, elaidic acid, palmitoleic acid, linoleic acid, α-linolenic acid, and γ-linolenic acid or combinations thereof. In certain embodiments, the fatty acid moieties have from 12 to 20 carbon atoms.

Further embodiments relate to compounds which are obtained by the variation of the hydroxyalkylamide fraction of the fatty acid amide compound, its homologue or its analogue. These embodiments include the introduction of a substituted or unsubstituted alkyl group of one to three carbon atoms (C1-C3) in the hydroxyl group of an alkanolamide or the remainder ethanolamide in order to form the corresponding lower alkyl ether. In another embodiment, the hydroxy group of the alkanolamide or ethanolamide moiety is attached to a carboxylate group of a substituted or unsubstituted (C 2 to C 6) alkyl group of alkyl carboxylic acid to form the corresponding ester of the fatty acid ethanolamide. Such embodiments include fatty acid alkanolamide and fatty acid ethanolamide amides on the ester linkage of organic carboxylic acids such as acetic acid, propionic acid and butanoic acid. In another embodiment, the fatty acid alkanolamide is oleoylalkanolamide.

In another embodiment, the fatty acid alkanolamide is oleoylethanolamide. In another embodiment, the fatty acid, homologous or analogous ethanolamide compound further comprises a substituted or unsubstituted (C3) alkyl group covalently attached to the nitrogen atom of the fatty acid ethanolamide.

The term “alkyl” refers to linear or branched hydrocarbon chain radicals having 1 to 10 carbon atoms, preferably 1 to 4, and which bind to the rest of the molecule by, for example, methyl, ethyl, n-propyl, i-propyl, n-butyl, tert-butyl, sec-butyl, n-pentyl, n-hexyl and the like. The alkyl groups may optionally be substituted by one or more substituents such as halogen, hydroxyl, alkoxy, carboxyl, carbonyl, cyano, acyl, alkoxycarbonyl, amino, nitro, mercapto and alkylthio.

The term “alkenyl” refers to hydrocarbon chain radicals containing one or more double carbon-carbon bonds, eg, vinyl, 1-propenyl, allyl, isoprenyl, 2-butenyl, 1,3-butadienyl, and the like. The alkenyl radicals may optionally be substituted by one or more substituents such as halo, hydroxyl, alkoxy, carboxyl, cyano, carbonyl, acyl, alkoxycarbonyl, amino, nitro, mercapto and alkylthio.

Compounds represented by formula (I), (II) or (III), may include isomers, depending on the presence of multiple bonds, including optical isomers or enantiomers, depending on the presence of chiral centers. The isomers, individual enantiomers or diastereoisomers and mixtures thereof fall within the scope, ie the term “isomer” also refers to any mixture of isomers, such as diastereomers, racemic, etc., even to their optically active isomers or the mixtures in different proportions thereof. The individual enantiomers or diastereoisomers, as well as mixtures thereof, may be separated by conventional techniques.

Also, within the scope are the prodrugs of the compounds of formula (I). The term “prodrug” or “prodrug” as used herein includes any derivative of a compound of formula (I) example and not limitatively: esters (including esters of carboxylic acids, esters of amino acids, esters of phosphate, sulphonate esters of metal salts, etc.), carbamates, amides, etc., which can be directly or indirectly transformed into said compound of formula (I) in said individual when administered to an individual. Advantageously, the derivative is a compound that increases the bioavailability of the compound of formula (I) when administered to an individual or potentiates the release of the compound of formula (I) into a biological compartment. The nature of the derivative is not critical so long as it can be administered to an individual and provides the compound of formula (I) in a biological compartment of an individual. The preparation of such prodrug may be carried out by conventional methods known to those skilled in the art.

As used herein, the term “derivative” includes both pharmaceutically acceptable compounds, i.e., derivatives of the compound of formula (I) as used in the liposome technology described above, it may also be used in the manufacture of a medicament, drug, dietary supplement, medical food or nutraceutical as pharmaceutically unacceptable derivatives, As these may be useful in the preparation of pharmaceutically acceptable derivatives.

The compounds may be in crystalline form as free compounds or as solvates. In this regard, the term “solvate” as used herein includes both pharmaceutically acceptable solvates, i.e., solvates of the compound of formula (I), for use with the liposome technology, it may also be used in the manufacture of a medicament, drug, dietary supplement, medical food or nutraceutical as pharmaceutically unacceptable solvates, which may be useful in the preparation of solvates or pharmaceutically acceptable salts. The nature of the pharmaceutically acceptable solvate is not critical as long as it is pharmaceutically acceptable. In a particular embodiment, the solvate is a hydrate. Solvates can be obtained by conventional solvation methods known to those skilled in the art.

For use in therapy, the compounds of formula (I), their salts, prodrugs or solvates, will preferably be in a pharmaceutically acceptable or substantially pure form, i.e., having a pharmaceutically acceptable level of purity excluding the additives. Pharmaceuticals such as diluents and carriers, and not including material considered toxic at normal dosage levels. The levels are preferably greater than 50%, more preferably greater than 70%, and still more preferably greater than 90%. In an embodiment, they are greater than 95% of the compound of formula (I), or salts, solvates or prodrugs thereof.

In another embodiment, alcohol use or consumption disorders are selected from alcohol intoxication or pathological intoxication, and alcohol dependence syndrome. In another embodiment, the alcohol use disorder is alcohol intoxication or pathological intoxication. Examples provide evidence of OEA-induced anti-inflammatory and neuroprotective effects. The results indicate that OEA interferes with the neuroimmune hazard signal HMGB1/TLR4/MyD88 associated with the NF-κB-mediated pro-inflammatory cascade and protects against hyperactivity of the proapoptotic caspase-3 enzyme in the rat frontal cortex, all caused by ethanol poisoning. In addition, OEA inhibited the activation of the hypothalamic-pituitary-adrenal axis (HPA) under exposure to alcohol intoxication without alteration in the metabolism of ethanol.

In another embodiment, the disorders or symptoms are selected from the list, which consists of neuro-inflammation, neurotoxicity, neuronal death, liver damage, vasalgia or any combination thereof. In another embodiment, the use or consumption disorder of alcohol is the alcohol dependence syndrome. In another embodiment, the disorders or symptoms are selected from the list consisting of neuro-inflammation, neurotoxicity, neuronal death, liver damage, vertigo, anhedonia, compulsion, tolerance and inability to controlling alcohol consumption, withdrawal from anxiety and depression, or any combination thereof.

The disorder or symptom associated with the alcohol dependence syndrome is neuro-inflammation, neurotoxicity, neuronal death, hepatic impairment and/or liver damage, or any combination thereof. The disorder or symptom of use or consumption of alcohol may be hangover or veasalgia. The disorder or symptom associated with alcohol dependence syndrome may be anhedonia. The disorder or symptom associated with alcohol dependence syndrome 30 may be compulsion, tolerance and/or inability to control alcohol consumption. The disorder or symptom associated with alcohol dependence syndrome may be anxiety. The symptom disorder associated with alcohol dependence syndrome may be depression.

One of the main conclusions of the examples has been that the pretreatment with OEA affected the expression and signaling receptors of 5 innate TLR4 immunity under conditions of alcohol intoxication. Therefore, in another embodiment of this aspect, the use is preventive and the administration of the compound is done prior to the ingestion of alcohol. In another embodiment, the use is preventive and the administration of the compound is made during the ingestion of alcohol.

The examples also show that ethanol intoxication induced overexpression and caspase-3 activity in the frontal cortex, which was inhibited by OEA pretreatment. This discovery reveals a mechanism not described to date that OEA can be used to protect the brain. Of particular interest are the data provided by behavioral experiments, 15 which indicate that OEA can regulate behavioral negatives associated with early states of alcohol withdrawal. Thus, OEA showed antidepressant-like properties in the forced swimming test 48 hours after alcohol abuse and a tendency to counteract alcohol-induced anxiety 24 hours after intoxication. The pattern of anxiety modulation was less pronounced 12 days after treatments. Therefore, another embodiment relates to the use of the compounds for use with the liposome technology. It may also be used in the manufacture of a medicament, drug, dietary supplement, medical food or nutraceutical for the prevention, relief, amelioration and/or treatment alcohol-induced anxiety in early states of alcohol withdrawal.

The compounds regulate multiple physiological adaptations after ethanol abuse, including reduction of ethanol auto-reestablishment and relapse, ethanol-induced neuro-inflammation and brain damage, or attenuation of withdrawal symptoms after ethanol ingestion. Preferably the compounds are administered before or during alcohol abuse. “Alcohol use disorders” describe a wide range of conditions ranging from symptoms caused by alcohol intoxication or pathological intoxication, to those associated with alcohol dependence syndrome. All of them are classified in ICD-10 under section F.10. Mental and behavioral disorders due to alcohol use, including:

F10.0 Acute intoxication
F10.1 Harmful use or harmful use
F10.2 Dependency syndrome
F10.3 Withdrawal status
F10.4 Withdrawal status with delusional
F10.5 Psychotic disorder
F10.6 Amnestic syndrome
F10.7 Residual and late onset psychotic disorder
F10.8 Other mental and behavioral disorders
F10.9 Mental and behavioral disorder, unspecified

Veisalgia, commonly known as a hangover, is a picture of general discomfort that occurs after an excessive consumption of alcoholic beverages, but not enough to reach deep coma and subsequent death from respiratory depression. It manifests itself as a set of the following symptoms such as light amnesia or loss of memory of what happened during the ethyl episode. It may include gastric disorders such as vomiting, almost always, and more rarely diarrhea because alcohol causes erosion of the gastric mucosa and loss of intestinal villi. Thus, liposomal technology with the PEA or related technology is applicable.

Other symptoms may include headache or headache, which is produced by dehydration of the meninges, dilation of the blood vessels and decrease of glucose (blood sugar), orthostatism and intense thirst, which originates as a response of the body to dehydration caused by the degradation of alcohol. Other symptoms may include abdominal and muscular pain, which results in a feeling of weakness, possible flatulence and nervous dullness.

“Alcohol” is understood herein, primarily, but not limited to, alcoholic beverages containing ethanol. Other alcohols that cause the same symptoms after ingestion are also possible. In an embodiment, the disease caused by excessive alcohol intake may be acute alcohol intoxication. In another embodiment, the disease caused by excessive alcohol intake may be mental and behavioral disorders (F.10 according to the ICD-10 classification). In another embodiment, the mental and behavioral disorder may be a psychiatric syndrome associated with alcoholism. The mental and behavioral disorder may be anxiety. In another embodiment, the mental and behavioral disorder may be depression.

In an embodiment the use or consumption disorder of alcohol is the alcohol dependence syndrome. In an embodiment, alcohol use disorders (or symptoms) are selected from the group consisting of neuro-inflammation, neurotoxicity, neuronal death, hepatic impairment, vertigo, anhedonia, compulsion, tolerance, and inability to control alcohol consumption, abstinence comprised by anxiety and depression, or any combination thereof. The alcohol dependence disorder (or symptom) associated with the alcohol dependence syndrome may be neuro-inflammation, neurotoxicity, neuronal death, hepatic impairment and/or liver damage, or any combination thereof.

The alcohol consumption disorder (or symptom) associated with alcohol dependence syndrome may be anhedonia. The alcohol consumption disorder (or symptom) associated with alcohol dependence syndrome may be compulsion, tolerance and/or inability to control alcohol consumption. The alcohol consumption disorder (or symptom) associated with the alcohol dependence syndrome may be anxiety. The alcohol consumption disorder (or symptom) associated with alcohol dependence syndrome may be depression. In another embodiment, the use is preventive and the administration of the compound may be done prior to the ingestion of alcohol.

In another embodiment, the use is preventive and the administration of the compound with the liposomes is made during the ingestion of alcohol. In another embodiment, the use is preventive and the administration of the compound with the liposome formulation is performed prior to or during the ingestion of alcohol. The composition may comprise as the only active ingredient a compound such as an active ingredient or another active ingredient. Pharmaceutically acceptable adjuvants and vehicles that may be used in such compositions are the adjuvants and carriers known to those skilled in the art.

As used herein, the term “therapeutically effective amount” refers to the amount of the agent or compound capable of developing the therapeutic action determined by its pharmacological properties, calculated to produce the desired effect and, in general, will be determined, among other causes, by the characteristics of the compounds, including age, patient status, severity of the disorder or disorder, and route and frequency of administration and the amount of inclusion in the liposome formulation.

The compounds described their salts, prodrugs and/or solvates as well as the pharmaceutical compositions containing them may be used in conjunction with other additional drugs or active agents to provide a combination therapy. Such additional drugs may form part of the same pharmaceutical composition or, alternatively, may be provided in the form of a separate composition for simultaneous or non-simultaneous administration of the pharmaceutical composition which comprises a compound of formula (I), or a salt, prodrug or solvate thereof. Thus, in another embodiment, the pharmaceutical composition further comprises another active ingredient. More preferably, the active ingredient is selected from the list consisting of vitamin E, vitamin C, betaine, N-acetylcysteine, ursodeoxycholic acid, resveratrol, hydroxytyrosol, lycopene and other antioxidants, nanoelectrolytes, minerals, probiotics, paracetamol, ibuprofen, vitamin B12, caffeine or any of its combinations used with the liposome formulation.

As used herein, the term “active ingredient,” “active substance or substance,” “pharmaceutically active substance or substance,” “active ingredient” or “pharmaceutically active ingredient” means any component which potentially provides a pharmacological or other effect. In diagnosis, cure, mitigation, treatment or prevention of a disease, or affecting the structure or function of the body of man or other animals. The term includes those components that promote a chemical change in drug manufacture and are present therein in a predicted modified form that provides the specific activity or effect.

The administered amount of a compound will depend upon the relative effectiveness of the compound selected, the severity of the disease to be treated and the patient's weight. However, the compounds of this invention will be administered one or more times a day with the liposomal formulation, for example, 1, 2, 3 or 4 times daily with a total dose between 0.1 and 1,000 mg/kg/day. It is important to note that dose variations may be necessary, depending on the patient's age and condition, as well as changes in the route of administration as noted above. The compounds and compositions may be used in conjunction with other drugs in combination therapies. The other drugs may form part of the same composition or of a different composition for administration at the same time or at different times.

A third aspect relates to a food composition or a composition nutraceutical composition or a composition of the “medical food” type, now referred to as the food composition comprising at least one of the compounds of formula (I), of formula (II) or of formula (III) and used with the liposome formulation. The liposome formulation may be added with food compositions that are selected from the group consisting of isotonic drinks, electrolyte-based preparations, juices, milk, yogurt, cheese, fermented milk, flavored milk beverage, soy milk, precooked cereals, bread, cakes, butter, margarine, sauces, frying oils, vegetable oils, corn oil, olive oil, soybean oil, palm oil, sunflower oil, cottonseed oil, condiments, salad dressings, fruit, syrups, desserts, frostings and fillings, soft frozen products, candy, chewing gum, thistle compositions, and intermediate foods.

Excipients may include, but are not limited to, starch, glucose, fructose, lactose, sucrose, gelatin, malt, rice, flour, calcium sulfate, silica gel, sodium, glycerol monostearate, talc, sodium chloride, skim milk powder, glycerol, propylene, glycol, water, ethanol, and the like. Such nutritional supplements may be used to combat liver problems, and help maintain health or a healthy lifestyle for the mammal, preferably a human.

There now follows examples of treatment of ethanol intoxication and using OEA as Example 10. Rats received an initial dose of 5 g/kg of 30% (w/v) ethanol via oral probe and a maximum of 3 g/kg of ethanol in subsequent doses every 8 hours. Blood was collected from the tail vein 2 hours before and 2 hours after 15:00 hours of ethanol administration by gavage on days 2 to 4 in order to determine blood ethanol levels (BEL), and treated of keeping toxic ethanol doses BEL relatively constant (see the table below). Mean ethanol/rat doses were 8 g/kg, 7.5 g/kg, 7.9 g/kg and 4.9 g/kg, day 1 to 4, respectively, and the mean ethanol/rat/day (1-4) was 7.06 g/kg. In a first experiment, the time-course of the neuro-inflammation was verified after 1 hour, 6 hours and 24 hours from the last feeding forced with ethanol. In the second experiment, OEA (5 mg/kg, ip, 10 mg/kg loading dose) was injected as pretreatment 10 min before each ethanol forced feed, and brain/blood tissue samples were collected 2-4 hours after the last administration of ethanol.

The animals were treated with intragastric (e.g.) ethanol 3 times a day using a cannula, i.e., (16G needle, Fisher Scientific, Waltham, Mass., USA), following a protocol based on a slightly modified standard paradigm of 4 days of alcohol intoxication. Ethanol doses were given every 8 hours for a total time of 4 days. Ethanol-treated rats received an initial loading dose of 5 g/kg ethanol in a 30% (w/v) solution and then a maximum of 3 g/kg maintenance dose, which were determined on the basis of levels of ethanol in blood, from English “blood ethanol levels” (BEL). This paradigm of repeated pattern of alcohol intoxication maintained a relatively constant BEL intoxication in a range of sedation/ataxia (190 to 430 mg/dl), according to a six-point alcoholic intoxication behavior scale. The mean dose of ethanol/rat/day (days 1-4) was 7.06 g/kg.

| | Hour When | | | | |
|---|---|---|---|---|---|
| | Blood | Day 2 | Day 3 | Day 4 | |
| Treatment | Collected | BEL (g/dL) | BEL (g/dL) | BEL (g/dL) | |
| Vehicle + | 13:00 | 193.29 ± 38.22 | 341.73 ± 32.48** | 303.32 ± 37.17* | N = 10 |
| EtOH | 17:00 | 274.96 ± 27.17 | 422.35 ± 18.00*** | 430.31 ± 19.72*** | N = 10 |
| OEA + | 13:00 | 217.14 ± 41.58 | 342.57 ± 40.25* | 320.68 ± 36.63 | N = 9 |
| EtOH | 17:00 | 321.88 ± 34.08 | 404.17 ± 27.65 | 429.76 ± 29.25* | N = 9 |

The data in the table above represents BEL 2 hours before and 2 hours later from the 15:00 hour of the administration of alcohol to animals in vehicle or pretreated with OEA. There were differences in BEL between day 2 and day 3 or 4 of treatment in both groups. Pretreatment with OEA did not modify BEL achieved during days 2-4 of alcohol intoxication treatment either before or after forced ethanol feed. Results are expressed as means±S.E.M. It is different from day 2 in the same treatment group: * p<0.05; ** P<0.001. [EtOH: ethanol; OEA: oleoylethanolamide; BEL: levels of ethanol in blood].

Determination of blood ethanol levels. To check for intoxication, BEL was determined on blood samples taken from the glue 120 minutes before and after the second administration, i.e., of ethanol of the day, which was made at 3 pm by the use of electrochemical detection of an enzymatic reaction with an alcohol analyzer AM1 (Analox Instruments, London, UK).

Experimental design, drug administration and tissue/plasma collection are now discussed. The animals (n=22) were sacrificed 1 hour, 6 hours, and 24 hours after the last administration of ethanol using a lethal dose of pentobarbital sodium (300 mg/kg, ip, Dolethal®, Spain). The brains were separated from the skull, and the meninges and blood vessels were carefully discarded. The frontal cortex was excised and frozen at −80° C. until the test. Blood was collected by cardiac puncture using trisodium citrate (3.15% w/v) as anticoagulant. Plasma was obtained by centrifugation of blood (2000 g) for 15 minutes at 4° C. and stored at −20° C. until determinations.

In this first experiment, the moment of expression of the main markers of inflammation were observed. Accordingly, a second experiment was designed in which samples of brain tissue were collected within a range of 2-4 hours after the last ethanol intoxication. This interval was chosen between 1 and 6 hours to detect both early changes in inflammatory parameters, e.g., overexpression of TNF-α, such as activation of posterior markers, such as expression of COX-2.

Effects of repeated oral OEA on alterations in neuro-inflammatory markers induced by exposure to alcoholic intoxication. In this second experiment (n=40 animals) the aim was to test the OEA anti-inflammatory/neuroprotective effects on the TLR4 signaling cascade in a model of alcohol abuse intoxication.

HMGB1 is a dangerous cytokine recruited by alcohol that activates the MyD88-dependent TLR4 immune signaling pathway, inducing the translocation of the p65 subunit of NF-kB to the nucleus and increasing its transcriptional activity. The NF-kB-mediated pro-inflammatory cascade involves the release of cytokines, such as TNF-α and IL-1β, which induce increased NF-kB activation and further neuro-inflammation, and chemokine MCP-1 (which is also induced by an alteration of oxidative stress), which mediates recruitment in the microglia and increases neurodegeneration. The transcriptional activity of NF-kB induces the expression of other pro-inflammatory markers, such as COX-2 and iNOS, leading to oxidative and nitrosative stress. Lipid peroxidation, measured by the accumulation of 4-HNE, induces a redox cell state related to activation of caspase 3 and cell death by caspase-mediated apoptosis 8. OEA (5 mg/kg ip, except an initial dose of 10 mg/kg ip, was synthesized and dissolved in the vehicle (5% Tween-80 in saline) and injected 10 minutes before each of the administrations of ethanol by tube.

Intragastric effects are now discussed. Reference is made to the anti-inflammatory activity of the OEA at 10 mg/kg intraperitoneally in the frontal cortex prior to LPS injection. In this study, OEA was injected repeatedly as a pretreatment before each administration of ethanol. The OEA doses chosen in this study are in the dose range tested as pharmacologically active (5-20 mg/kg, i.p.). A time curve (n=18) with OEA (3 hours, 6 hours and 24 hours) was also performed to rule out any effects in the control animals.

Tests were conducted to determine the effects of repeated OEA administration on depressive and anxiety behavior during early ethanol abstinence. Animals were tested in the cross-raised labyrinth 24 hours and 12 days after the alcohol abuse protocol and forced swimming test was performed 48 hours after the last administration of ethanol. [HMGB1 (High Mobility Group Box 1): high mobility group 1 box; TLR4 (Toll-Like Receptors 4): Receptors type Toll 4: MD2 (Myeloid Differentiation Protein 2): myeloid differentiation protein 2; MyD88 (Myeloid Differentiation Factor 88): myeloid differentiation factor 88; NF-kB (Nuclear Farctor kB): nuclear transcription factor kappa B (p65 subunit); IkB: inhibitory protein IkappaB; TNF-α (Tumor Necrosis Factor α): tumor necrosis factor alpha; IL-1β (Interleukin β): 1interleukin-1 beta; MCP-1 (Monocyte Chemoattractant Protein 1): monocyte chemotactic protein 1; INOS (inducible Nitric Oxide Synthase): inducible nitric oxide synthase; COX-2 (Cyclooxygenase 2): cyclooxygenase 2; 4-HNE (4-hydroxynonenal): 4-hydroxynonenal].

In this third experiment (n=25), the treatment with ethanol intoxication and OEA pretreatment applied in experiment 2 was repeated, and the animals were submitted to the maze test in a high cross 24 hours and 12 days after the last forced ethanol administration and the forced swimming test 48 hours after the ethanol intoxication protocol.

There was a preparation of nuclear and cytosolic extracts. Nuclear and cytosolic protein extracts were obtained according to figures in the publications, Sayd et al., Systemic Administration of Oleoylethanolamide Protects from Neuroinflammation and Anhedonia Induced by LPS in Rats, Int. J. Neuropsychopharmacol (2014).

The western blot analysis was studied. The frontal cerebral cortex was homogenized by sonication in 400 μl of PBS (pH=7.4) mixed with a protease inhibitor cocktail (Complete, Roche®, Madrid, Spain), followed by centrifugation at 12,000 g for 10 minutes at 4° C. Homogenized with adjusted protein levels were mixed with Laemmli sample buffer (BioRad®, CA, USA) containing β-mercaptoethanol (50 μl/ml Laemmli) and 1 mg/ml were loaded onto an electrophoresis gel. Proteins were transferred to a nitrocellulose membrane (Amersham Ibérica®, Spain) with a semi-dry transfer system (Bio-Rad®, CA, USA), incubated with specific primary and secondary antibodies and revealed by the ECL™ kit (Amersham Ibérica®, Spain). Autoradiography was quantified by densitometry (NIH ImageJ® software, National Biosciences, Lincoln, Nebr. USA) and expressed as optical density (O.D.). In all Western blot analyzes, the β-actin protein was used as a loading control.

Real-time Polymerase Chain Reaction Analysis (RT-PCR) were studied. Total cytoplasmic RNA was prepared from frontal cortex samples using the TRIZOL® reagent (Invitrogen, Grand Island, N.Y., USA). Aliquots were converted to cDNA using random hexamer primers. Quantitative changes in mRNA levels were estimated by RT-PCR, carried out in the presence of the SYBR green probe using a 20-L reaction in a Rotor-Gene (Corbett Research®, Mortlake, NSW, Australia).

Levels of HMGB1, pro-inflammatory cytokines (TNF-α, IL-1β) and chemokines MCP-1 were studied. Levels of HMGB1, TNF-α, IL-1β and soluble MCP-1 were determined by Enzyme-linked immunosorbent assay (ELISA) 30 (Elabscience Biotechnology Co., Ltd., China, for HMGB-1; RayBiotech®, GA, USA, for the TNF-α, IL-1β and MCP-1). The nuclear/cytosolic HMGB1 fraction was determined by Western blot analysis.

Transcription factor assay NF-κB was studied. Nuclear extracts were used for the determination of NF-κB transcription factor activity by use of an ELISA-based kit (Cayman Chemicals©, Tallinn, Estonia). Nuclear extracts from the frontal cortex were incubated with probes specific for response elements to the p65 NF-kB subunit, and binding of p65 to its probe was detected using a specific antibody against this subunit. A horseradish peroxidase-labeled secondary antibody was added and the binding detected spectrophotometrically at 450 nm. The measurement was performed according to the manufacturer's instructions. This assay is specific for the activation of p65, and does not cross-react with other subunits of NF-kB, such as p50. The data were normalized by the amount of total protein.

Lipid peroxidation was studied. Lipid peroxides are unstable indicators of oxidative stress in cells that break down to form more complex and reactive compounds, such as 4-hydroxynenal (4-HNE), a natural byproduct of lipid peroxidation. Levels of HNE protein adducts in cerebral cortex lysates were measured using a competitive 96-well OxySelect™ adduct HNE ELISA Kit (Cell Biolabs®, San Diego, Calif., US).

Measurement of caspase-3 activity was studied. Caspase-3 activity was determined using a fluorometric assay kit (Abnova®, Taiwan) according to the manufacturer's protocol. This commercial kit allows the measurement of DEVD-dependent caspase activity.

Plasma levels of corticosterone and LPS were studied. Corticosterone and LPS were measured in plasma using commercially available kits by radioimmunOEAsay (RIA) (Coat-A-Count®, Siemens, La., USA) or colorimetric enzyme reaction (HyCult Biotech, Uden, respectively).

Protein assay was studied. Protein levels were measured using the Bradford method based on the principle of the dye binding protein.

Behavioral tests were studied. The forced swimming test was performed on the rats two days after the last forced feed with ethanol and the specific behaviors were recorded with a digital video camera for 5 minutes. These same rats were also tested by the high cross maze test equipped with infrared light (Panlab, Barcelona, Spain) 1 and 12 days after the last forced feed with ethanol, allowing the animals to freely explore the labyrinth during 5 minutes. Tests were performed 3 hours after onset of the dark phase.

Statistic analysis was studied. Data are expressed as mean±SEM. The data were analyzed (ANOVA), comparing factors 15 [alcohol/water] versus [OEA/vehicle], followed by a Bonferroni post hoc test, and by analysis of variance ANOVA of a path followed by the Newman-Keuls post hoc test when necessary. The BEL data were analyzed by repeated measures of analysis of the 2-way ANOVA variance followed by a Bonferroni post-hoc test (treatment×daily determination), both for the BEL pre and post forced feed 20 probe determinations. The behavioral test data were examined to determine normal conditions with the Kolmogorov-Smirnov test and then analyzed by parametric two/one-way ANOVA of one factor followed by post hoc Bonferroni/Newman-Keuls tests (forced swimming test) or non-parametric Kruskal-Wallis ANOVA followed by the Dunn's post hoc test for multiple comparisons (high labyrinth 25) where appropriate. A value of p≤0.05 was considered statistically significant. Data were analyzed using GraphPad Prism version 5.04 (GraphPad Software Inc., San Diego, Calif., USA).

The description of example 11 now follows. Temporal curve of pro-inflammatory markers in frontal cortex induced by alcohol intoxication and increase of plasma corticosterone levels. The parameters were measured 1 hour, 6 hours and 24 hours after the last administration of ethanol. TNF-α levels were overexpressed in the frontal cortex 6 hours after treatment with excess ethanol and were infra-expressed 24 hours after treatment. The levels of IL-1β showed a tendency to increase after treatment with excess ethanol, but were not significantly elevated. The activity of the p65 subunit of NFκB was increased and decreased in the nuclear extracts of the frontal cortex 6 hours and 24 hours after the excess of ethanol, respectively. IκBα levels were elevated at all times after treatment with excessive ethanol. The enzyme COX-2 is overexpressed 6 hours after exposure to ethanol. Treatment with excess ethanol increases blood corticosterone levels 1 hour, 6 hours, and 24 hours after the last forced feeding with ethanol. The data represent the mean±S.E.M. (N=3-6). Different from the control group: * p<0.05; ** p<0.01; *** p<0.001.

TNF-α from the frontal cortex was overexpressed 1 hour after administration of ethanol [55% increase over control (80.29±17 pg/mg protein)] and cytokine content decreased 24 hours After treatment (F (3.11)=9.45; P=0.0002). IL-1β showed a tendency to increase 1 h after the exposure to ethanol [29% increase over control (75.5±6.6 pg/mg protein)], but the data did not become statistically significant.

The expression of the p65 subunit of NF-kB was increased in the nuclear extracts of the frontal cortex 6 hours after treatment with ethanol, and decreased 24 hours after ethanol intoxication, when compared with the control group (F (3.11)=20.48, p<0.0001). Levels of the NF-kB inhibitory protein, IkappaB-alpha (IκBα), remained elevated 1 hour, 6 hours and 24 hours after treatment with alcohol in the cytosolic extract of the frontal cortex (F (3,13)=6, 73, p=0.0056), and the COX-2 enzyme showed an overexpression of 6 hours after ethanol administration (F (3.13)=3.69; p=0.04). Finally, the exposure to alcoholic intoxication raised corticosterone levels in plasma 1 hour, 6 hours and 24 hours after the last administration (F (3.15)=3.91, p=0.03). Based on this pattern of expression of the major pro-inflammatory markers between 1 hour and 6 hours after the last administration of ethanol by intragastric catheter, we chose to extract brain/plasma samples within 2-4 hours after protocol of intoxication to study the pharmacological effects of the OEA.

### Example 12

Levels of Blood Ethanol. It was studied whether the OEA could modify BEL during days 2-4 of excess alcohol treatment. The repeated measures two-way ANOVA detected differences in BEL throughout the days of intoxication protocol (F (2, 17)=10.17; p=0.0003; F (2, 17)=15.41, P<0.0001), and demonstrated that pretreatment with OEA did not alter the EF achieved during days 2-4 of treatment with ethanol intoxication, nor in forced pre-feeding (F (1,17)=0, 13, p=0.72, ns) or in the post-feed (F (1,17)=0,18, p=0.67, ns). BEL also determined just before tissue removal (day 5), with 431.98±34.90 g/dl for the vehicle+ethanol group and 445.82±32.70 g/dl for the OEA+ethanol group (Student t-test, P=0.78, ns).

### Example 13

Alterations of the HMGB1/TLR4 signaling pathway and the effect of the OEA pretreatment on the excessive consumption of ethanol. In order to explore whether pro-inflammatory mediators such as cytokines or NF-κB were secreted by the activation of innate immunity receptors induced by ethanol, the influence of ethanol intoxication on the expression of TLR4 and its endogenous activator, the hazard signaling cytokine HMGB1. In addition, the co-receptor TLR4 was tested, myeloid differentiation protein 2 (MD2), and expression of myeloid differentiation factor 88 (MyD88), which is an adapter protein for the intracellular transduction signaling cascade TLR4.

The principal effects on ethanol exposure and pretreatment with OEA 5 on the expression of HMGB1 was observed. The increase in HMGB1 induced by ethanol intoxication (F (1.13)=19.14, p=0.0008) was prevented by OEA pretreatment (FF (1.13)=4.58, P=0.05). Analysis of the cytoplasmic/nuclear HMGB1 ratio revealed that the total increase in HMGB1 expression in animals exposed to ethanol may be due to an increase of cytokine in both nucleus and cytoplasm, 10 being maintained in rats treated with ethanol higher levels in the nucleus than in the cytoplasm (ratio n/c=1.21). OEA pretreatment decreased ethanol-induced HMGB1 expression more efficiently in the cytosolic fraction (ratio n/a=1.48), indicative of having activity on the release of HMGB1 from the nucleus to the cytoplasm in rather than at the level of synthesis.

Treatment with ethanol intoxication has a major effect on both TLR4 mRNA (F (1, 17)=10.08, p=0.005) and protein expression. 10.04, p=0.004), a major effect of OEA on TLR4 mRNA (F (1,17)=6.08, p=0.0246) and an interaction between exposure to ethanol and OEA (F (1, 24)=9.67, p=0.0048) at levels of the TLR4 protein. The pretreatment with OEA was able to reduce the overexpression of (3, 17)=5.86, p=0.0061) and protein expression (F (3.24)=8.15, P=0.0006).

Exposure to ethanol by intoxication increases the expression of the MD2 protein. F (1.27)=6.83, p=0.0145), but the mRNA expression data did not reach statistical significance (F (1.31)=3.79, p=0.06, ns). The main effect of the pretreatment with OEA (F (1, 27)=4.24, p=0.04) and one interaction (exposure to ethanol vs. pretreatment) (F (1,17)=4.57, p=0.0418) were also found at MD2 protein levels. Pretreatment with OEA inhibited the increase in MD2 induced by ethanol (F (3.30)=5.52, p=0.044).

Ethanol intoxication induced a major effect on MyD88 mRNA (F (1, 15)=10.34, p=30 0.0058, data not shown) and protein expression (1.30)=9, 55, p=0.0043) and an interaction (ethanol×pretreatment) was also found at MyD88 protein levels (1, 30)=4,640, p=0.0394). Pretreatment with OEA inhibited the increase in ethanol-induced MyD88 protein expression (3, 32)=5.46, p=0.042).

### Example 14. Effects of OEA on Pro-Inflammatory

cytokines TNF-α and IL-1β and chemokines MCP-1 in frontal cortex and plasma. Treatment with excess ethanol increased the mRNA expression of both TNF-α and of IL-1β in the frontal cortex, although pretreatment with OEA did not prevent this effect. TNF-α protein levels were increased in the frontal cortex of rats under treatment with excess ethanol (F (1, 16)=2.56, p=0.1281) [24% increase over the values of the control (95.2±3.1 pg/mg protein), and were not avoided by OEA (FIG. 15A; F (3.16)=0.02, p=0.89, ns). In plasma, a strong increase in TNF-α levels was observed in the animals treated with ethanol (F (1,14)=44.64, p<0.0001) [141% 10 increase with respect to Values of control (113.0±5.1 pg/mg protein)] and an interaction between ethanol and pretreatment with OEA (F (1,14)=4.85, p=0.045). One-way ANOVA indicated that pretreatment with OEA partially prevented the increase of TNF-α in plasma observed after ethanol intoxication (F (3.17)=15.59, p<0.0001).

The animals treated with ethanol showed an increase in IL-1β protein levels in the frontal cortex (F (1.16)=4.497, p=0.0499) [27% increase over control values (79, 31±0.29 pg/mg protein), which was prevented by OEA pretreatment (F (1,16)=4.22, p=0.05). In plasma, an interaction between OEA pretreatment and ethanol administration was observed (F (1.20)=18.15, p=0.0004). Treatment with excess ethanol increased plasma levels of IL-1β [53% of 20 increase over control values (85.29±7.8 pg/mg of protein)], an effect that was prevented by OEA (F (3,20)=8.20, p=0.0009).

As for MCP-1 levels, there was an interaction between ethanol administration and OEA pretreatment (F (1.33)=10.06, p=0.0033) and the main effects of ethanol (F 1.33)=4.69, p=0.0377) and pretreatment with OEA (F (1.33)=12.88, p=0.0011). The treatment 25 with excess ethanol increased MCP-1 levels in the front cortex and pretreatment with OEA prevented this effect (F (3.33)=9.92, p<0.0001).

### Example 15

Effect of OEA on NF-κB expression and activity after exposure to ethanol intoxication. The pro-inflammatory canonical pathway of NF-kB in nuclear extracts and 30 cytosolic frontal cortex after pharmacological treatments were explored. There was a major effect of ethanol intoxication treatment at p65 mRNA levels (F (1.33)=44.14, p<0.0001) and an interaction between treatment with ethanol and pretreatment With OEA (F (1.33)=9.10, p=0.0049). One-way ANOVA revealed that pre-administration of OEA partially prevented the increase in p65 mRNA levels induced by ethanol (F (3.36)=19.87, p<0.0001). In the activity trial, there were major effects of alcohol exposure (F (1.16)=6.55, p=0.021) and pretreatment with OEA (F (1.16)=16.66, P=0.0009), the increase in nuclear activity of p65 induced by 5 ethanol counteracted by pretreatment with OEA.

Treatment with excess ethanol also increased the expression of mRNA and IκBα protein (1.35)=50.64, p<0.0001; F (1.33)=37.69, p<0.0001) and interacted with the pretreatment with OEA (F (1.33)=8.81, p=0.0055). Pretreatment with OEA did not counteract the increase in IκBα mRNA levels and protein expression induced by ethanol (F (1.35)=0.17, p=0.6848 ns, F (1.33)=0.17, p=0.067, p=0.7970, ns, respectively).

### Example 16

Effect of OEA pretreatment on the expression of iNOS and COX-2 mRNA and lipid peroxidation after exposure to ethanol intoxication. Activation of NF-κB culminates in the production of inflammatory mediators such as the 15 inducible iNOS and COX-2 enzymes. The ethanol intoxication model used in this study produced a strong increase in the expression of iNOS mRNA (F (1.17)=11.87, p=0.0031) which was completely prevented by the pretreatment with OEA (F (1.17)=8.71, p=0.0089). The maximum expression of COX-2 observed after ethanol treatment in the time-course experiment was carried out 6 hours after the end of the intoxication protocol, which could explain the absence of A significant effect of ethanol on the regulation of COX-2 mRNA in the second experiment, in which brain samples were collected 2-4 hours after the end of the last ethanol administration (F (1, 32)=0.07, p=0.7975, ns). However, 2-way ANOVA detected a main effect of pretreatment with OEA (F (1.32)=7.17, p=0.0011) and an interaction (ethanol×OEA) (F (1, 32)=6.94, p=0.0129). One-way ANOVA analysis revealed that the OEA reduced COX-2 mRNA levels with ethanol, but not in the animals treated with vehicle (F (3.32)=4.26, p=0.0123).

As a result of over-activation of the pro-inflammatory pathways, the cellular mechanisms of oxidative stress inducing the formation of lipid peroxides that will affect cell viability. The formation of the HNE adduct, a reactive compound formed by the decomposition of the lipid peroxides, was found in the experiments. An increase in HNE formation in the frontal cortex of animals exposed to ethanol intoxication was observed. F (1.15)=14.56, p=0.0017) and an effect of pretreatment with OEA (F (1.15)=7.21, p=0.017). OEA decreased the HNE formation, being the significant effect only in the animals treated with ethanol (F (3.18)=7.03, p=0.0036).

### Example 17

Effect of OEA on caspase-8 and proapoptotic caspase-3 following treatment with ethanol intoxication. It was studied whether the induction of the TLR4/NF-κB signaling cascade by ethanol culminated in neuronal damage. The expression and activity of the proapoptotic caspase-3 enzyme in the frontal cortex of animals under different drug treatments, and overexpression of caspase-8 was checked, which acts as an intermediate caspase in the TLR4-MyD88/Caspase-8/caspase-3.

Caspase-8 mRNA was overexpressed after ethanol intoxication (F (1.15)=13.37, p=0.023), and pretreatment with OEA reversed this effect F (3.18)=6, 95, p=0.037). Caspase-3 mRNA was overexpressed with alcohol, but OEA was not able to prevent this effect. As regards levels of the caspase-3 protein, an interaction between alcohol exposure and pretreatment with OEA was observed (F (1,15)=7.95, p=0.0129) and a major effect of ethanol (F (1.15)=12.30, p=0.0032). One-way ANOVA analysis indicated that pretreatment with OEA prevented ethanol-induced overexpression of caspase-3 (F (3.18)=7.29, p=0.0031). In addition, treatment with ethanol intoxication induced an increase in caspase-3 activity in the frontal cortex (F (1.32)=20.98, p<0.0001), which was reversed by pretreatment with OEA (FIG. 17F) (1.32)=4.774, p=0.0369).

### Example 18

Effect of OEA on Plasma Corticosterone and LPS Levels. To test the effects on the axis (HPA), the influence of the OEA on the increase of the levels of corticosterone induced by ethanol was tested. The data revealed a significant treatment effect (F (1.29)=5.44, p=0.00268) and an interaction between the ethanol poisoning and pretreatment with OEA (F (1,29)=4.77, p=0.0373). An increase in corticosterone levels was observed in animals exposed to ethanol intoxication treatment, which was completely prevented by pretreatment with OEA (F (3.29)=4.21, p=0.0136). OEA did not modify plasma corticosterone levels in control animals.

In addition, the effect of OEA pretreatment on intestinal permeability to endotoxins was assessed by checking plasma LPS levels after treatments. Treatment with ethanol intoxication significantly increases plasma LPS levels approximately 80% with respect to the control (F (1.27)=12.40, p=0.0015).

Animals that were pretreated with OEA and receiving ethanol showed lower LPS plasma levels (44% increase over control), a value that did not differ with either the control group or the ethanol group. Two-way ANOVA indicated that there was no significant pretreatment effect (F (1.27)=0.50, p=0.4872), and that there was no interaction between exposure to ethanol and pretreatment with OEA (F (1.27)=2.376, p=0.01348).

### Example 19

Effect of OEA on control animals. The OEA time curve in control animals indicates that OEA did not modify the parameters in the controls. However, since some trends are observed in comparison with control levels 3 hours post-injection (i.e., TLR4 and p65), it may not be possible to rule out completely that the OEA may affect control animals in the early stages.

### Example 20

OEA Effect on Symptoms of Type Depressive and Anxiety. In order to determine the effects of OEA on ethanol-related behaviors during alcohol withdrawal, the forced swimming test and the elevated cross-maze test were performed to test the symptoms of depressive and anxiety type respectively. The results showed that ethanol intoxication caused a reduction in swimming and climbing times, F (1,21)=3,281, p=0.05, F (1,21)=7,326, p=0.0132, respectively) and increased immobility and latency (1.21)=9.53, p=0.0056; F (1.21)=9.53, P=0.006, respectively) in the forced swimming test. An interaction between OEA treatment and ethanol intoxication was also observed in the immobility and latency parameters (F (1.21)=4.43, p=0.0475, F (1.21)=9.53, P=0.006, respectively). Pretreatment with OEA was able to prevent alcohol-induced depressive symptoms, normalizing the 25 measures of swimming, immobility, and latency. F (3.21)=2.935, p=0.05; F (3.21)=5.77, p=0.048; F (3.21)=4.17, p=0.018, respectively).

Regarding anxiety-type behaviors, the Kruskal-Wallis ANOVA test indicated that there was an overall effect of treatments on the percentage of entries (H=8.074, p=0.05) and on time (H=7.702, p=0.0526) of permanence in the open labyrinth arms raised 24 hours after ethanol intoxication. Although there was a clear trend showing a clear anxiolytic effect of OEA in the animals treated with ethanol and control, the Dunn post hoc test found no significant effects in multiple comparisons. The effect of the treatments on the same measures of anxiety, the percentage of entrances and the time of permanence in the open arms was not significant 12 days after ethanol intoxication (H=6,377, p=0.09 and H=5.619, p=0.13, ns, respectively).

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A liposomal rehydration salt formulation, comprising phospholipids at a concentration of about 1.0 g/L to 60.0 g/L, salts, water, and a percentage inclusion ratio of salts (salts retained within total salts/liposomes) of at least 40%, and a compound of general formula (I) 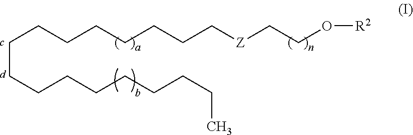
or any of its pharmaceutically acceptable salts, esters, tautomers, solvates and hydrates, or any combination thereof;
where:
n is an integer ranging from 0 to 5;
a and b are determined by the following formula: 0≤(a+b)≤4; and
Z is a group selected from —C(O)N(R0)-; —(R0)NC(O)—; —(O)CO—; OR; NR0; and S, and wherein R0 and R2 are independently selected from the group consisting of substituted or unsubstituted alkyl, hydrogen, substituted or unsubstituted (C1-C6) alkyl, substituted or unsubstituted (C1-C6) acyl, and aryl, and wherein up to eight hydrogen atoms of the compound may be substituted by methyl or a double bond, and the molecular bridge between c and d may be saturated or unsaturated, and
wherein said formulation comprises an osmolality lower than 190 mmol/L.

2. The liposomal rehydration salt formulation of claim 1, wherein said compound comprises oleoylethanolamide.

3. The liposomal rehydration salt formulation of claim 1, wherein said liposomes comprise a particle diameter ranging from 200 nm to 500 nm.

4. The liposomal rehydration salt formulation of claim 1, wherein said compound is in the range of about 0.1 to 200 mg per daily dosage serving of the formulation.

5. The liposomal rehydration salt formulation of claim 1, wherein said compound has a percentage inclusion ratio of total compound/liposomes of at least about 40 percent.

6. A liposomal rehydration salt formulation, comprising phospholipids at a concentration of about 1.0 g/L to 10.0 g/L, salts, water, oleoylethanolamide, and a percentage inclusion ratio of salts (salts retained within total salts/liposomes) of at least 50% and a sodium electrolyte of about 12 mEq/L to 90 mEq/L, and said liposomes comprise a particle diameter ranging from 200 nm to 500 nm and said formulation comprises an osmolality lower than 190 mmol/L.

7. The liposomal rehydration salt formulation of claim 6, wherein said oleoylethanolamide is in the range of about 0.1 to 200 mg per daily dosage serving of the formulation.

8. The liposomal rehydration salt formulation of claim 7, wherein said oleoylethanolamide is in the range of about 5 to 30 mg per daily dosage serving of the formulation.

9. The liposomal rehydration salt formulation of claim 6, wherein said oleoylethanolamide has a percentage inclusion ratio of total compound/liposomes of at least about 40 percent.

10. The liposomal rehydration salt formulation of claim 6, wherein said sodium electrolyte is from about 35 mEq/L to 55 mEq/L.

11. The liposomal rehydration salt formulation of claim 6, further comprising about 15 mEq/L to 25 mEq/L of potassium electrolyte.

12. The liposomal rehydration salt formulation of claim 6, wherein said phospholipids are selected from the group consisting of phosphatidylcholines (PCs), phosphatidylserines (PSs), phosphatidylethanolamines (PEs), phosphatidylglycerols (PGs), phosphatidylinositols (PIs), phosphatidic acids (PAs), and mixtures thereof.

13. The liposomal rehydration salt formulation according to claim 6, wherein said formulation further comprises an antioxidant selected from the group consisting of phytosterol, tocopherol, and mixtures thereof.

14. The liposomal rehydration salt formulation of claim 6, wherein said salts are selected from the group consisting of sodium chloride at a concentration of 0.7 g/L to 2.8 g/L, potassium citrate at a concentration of 0.8 g/L to 2.5 g/L, sodium citrate at a concentration of 0.5 g/L to 2.9 g/L, and mixtures thereof.

15. The liposomal rehydration salt formulation of claim 6, wherein said formulation further comprises about 10 g/L to 17 g/L of glucose and about 8.0 g/L to 15 g/L of at least one additional sugar.

16. A method of treating alcohol related disorders, comprising orally administering a liposomal rehydration salt formulation, comprising phospholipids at a concentration of about 1.0 g/L to 60.0 g/L, oleoylethanolamide, salts, water, and a percentage inclusion ratio of salts (salts retained within total salts/liposomes) of at least 40%, and wherein said formulation comprises an osmolality lower than 190 mmol/L.

17. The method of claim 16, wherein said liposomes comprise a particle diameter ranging from 200 nm to 500 nm.

18. The method of claim 16, comprising administering about 0.1 to 200 mg of said oleoylethanolamide per daily dosage serving of said formulation.

19. The method of claim 16, comprising administering about 5.0 to 30.0 mg of the oleoylethanolamide per daily dosage serving of said formulation.

20. The method of claim 16, wherein the oleoylethanolamide has a percentage inclusion ratio of total compound/liposomes of at least about 40 percent.
